(19) United States
(12) Patent Application Publication
(10) Pub. No.: US 2020/0197306 A1
(43) Pub. Date: Jun. 25, 2020

(54) CATIONIC LIQUID CRYSTALLINE NANOPARTICLES

(71) Applicant: Board of Regents, The University of Texas System, Austin, TX (US)

(72) Inventors: Emanuela GENTILE, Houston, TX (US); Ji LIN, Sugar Land, TX (US); Jack A. ROTH, Houston, TX (US)

(73) Assignee: Board of Regents, The University of Texas System, Austin, TX (US)

(21) Appl. No.: 16/620,090

(22) PCT Filed: Jun. 7, 2018
(86) PCT No.: PCT/US2018/036483

§ 371 Date: Dec. 6, 2019

Related U.S. Application Data
(60) Provisional application No. 62/516,629, filed on Jun. 7, 2017.

(51) Int. Cl. A61K 9/127; A61K 9/51; A61K 47/24; A61K 47/14; A61K 31/713; A61K 31/7105; A61K 9/00

(52) U.S. Cl. CPC A61K 9/1272; A61K 9/5192; A61K 47/24; B82Y 5/00; A61K 31/713; A61K 31/7105; A61K 9/0019; A61K 47/14

(57) ABSTRACT

Provided herein are cationic liquid crystalline nanoparticles (CLCNs). Further provided herein are methods of delivering RNAi using the CLCNs for the treatment of diseases.

The present application claims the priority benefit of U.S. Provisional Application Ser. No. 62/516,629, filed Jun. 7, 2017, the entire contents of which are hereby incorporated by reference.

This invention was made with government support under grant numbers CA176568 and CA070907 awarded by the National Institutes of Health. The government has certain rights in the invention.

### BACKGROUND

### 1. Field

The present disclosure relates generally to the fields of molecular biology and medicine. More particularly, it concerns compositions for the delivery of nucleic acids, such as RNA. Specifically, it concerns cationic liquid crystalline nanoparticles (CLCNs) for the delivery of nucleic acids, such as RNAi.

### 2. Description of Related Art

RNA interference (RNAi) is a potential new class of drugs that can selectively silence disease-causing genes, including those causing genetic disorders, viral infections, autoimmune diseases, and cancer. Two types of small RNA molecules are central to RNA interference: small interfering RNA (siRNAs) and microRNA (miRNAs) inhibitors and mimics. Current efforts to introduce RNAi usage in the clinic involve the development of safe and effective systemic delivery systems that are stable in circulating blood and induce efficient cellular uptake. Based on the natural process of cell infection and the transfer of genetic materials into host viruses have been evaluated as possible gene carriers, but toxicity, immunogenicity, and the inadequate size of the inserted genetic materials impair their efficacy in vivo. Thus, while current RNAi delivery systems may be effective in vitro, they have been shown to have limited efficacy and stability in vivo combined with high toxicity.

To overcome these challenges, non-viral vectors such as lipid-based delivery systems, cationic liposomes, lipid nanoparticles, and a variety of cationic and biodegradable polymers have been used to mask the negative charges of the siRNA or miRNA backbone and facilitate cellular uptake, partially mediating the efficient delivery of siRNA in vitro and in vivo. However, there is an unmet need for efficient delivery systems which are non-toxic and stable in circulation.

### SUMMARY

In some embodiments, the present disclosure provides cationic liquid crystalline nanoparticles (CLCN) including glycerol monooleate (GMO), a cationic phospholipid, and a nonionic surfactant. The nonionic surfactant may be present in a concentration of up to 5% by weight. The nanoparticle may be positively charged.

The CLCNs may have one or more of the following additional features, which may be combined with one another unless clearly mutually exclusive: a) the nanoparticles may include a lipid bilayer enclosing an aqueous core in which the bilayer is surrounded by a hydrophobic shell; b) the nonionic surfactant may be present at a concentration of 0.1 to 1% by weight, such as 0.5% by weight; c) the nonionic surfactant may be a nonionic polyol, such as tri-block polyethylene glycol-polypropylene-polyethylene glycol, such as Pluronic F-127; d) the nanoparticle may have a zeta potential of +25 to +35 mV, such as greater than +30 mV: e) the nanoparticle may have a diameter of 60 to 100 nm, such as less than 100 nm, 90 nm, 80 nm, or 70 nm; f) the cationic phospholipid may be 2-dioleoyl-3-trimethylammonium-propane chloride salt (DOTAP), Dimethyldioctadecylammonium (DDAB), 1,2-di-O-octadecenyl-3-trimethylammonium propane (DOTMA), 3ß-[N—(N′,N′-dimethylaminoethane)-carbamoyl]cholesterol hydrochloride (DC-CHOL), 1,2-dioleoyl-3-dimethylammonium-propane (DODAP), cetyl trimethyl ammonium bromide (CTAB), 1,3-dioleoyloxy-2-(6-carboxy-spermyl)-propyl-amide (DOSPER), or 1,2-dioleoyl-sn-glycero-3-[(N-(5-amino-1-carboxypentyl)iminodiacetic acid)succinyl](DOGS); g) the cationic phospholipid may be present at a concentration of less than 30% by weight, such as less than 20% or 15% by weight, particularly 5-10% by weight; h) the glycerol monooleate may be 1-(cis-9-octadecenoyl)-rac-glycerol; i) the nanoparticles may have a polydispersity index (PDI) of 0.10 to 0.20: j) the nanoparticles may be loaded with RNA, such as siRNA or miRNA; k) the RNA may have a length of 15 to 50 nucleotides: 1) the RNA may be loaded in the nanoparticles at a 1:1 volume ratio; m) the RNA may be encapsulated within the nanoparticles; n) the zeta potential of the RNA-loaded nanoparticles may be between +30 to +45 mV.

Another embodiment provides a composition comprising a plurality of the nanoparticles provided herein. The nanoparticles may have a median mass aerodynamic diameter (MMAD) of 60-100 nm.

Further embodiments provide pharmaceutical compositions comprising a plurality of the nanoparticles provided herein in combination with a pharmaceutically acceptable carrier.

In another embodiment, there is provided a method of producing cationic liquid crystalline nanoparticles (CLCNs). The method may comprise solubilizing one or more cationic phospholipids and glycerol monooleate in ethanol to obtain a lipophilic stage:

solubilizing a nonionic surfactant in water to obtain a hydrophilic phase; emulsifying the lipophilic phase and the hydrophilic phase using high-speed homogenization to obtain a nanoparticle solution: and evaporating the ethanol from the nanoparticle solution, thereby obtaining CLCNs. The CLCNs may be the CLCNs described above, including all combination of features.

The method of production may comprise one or more of the following features: a) the nonionic surfactant may be Pluronic F-127; b) the emulsification step may comprise dropwise addition of the hydrophilic phase to the lipophilic phase, wherein the lipophilic phase is under high-speed homogenization: c) the high-speed homogenization may be at a speed of 7,000-10,000 rpm or 10.000-20,000 rpm; d) the method may further comprise applying the CLCN solution produced from the emulsification step to one or more rounds of high-speed homogenization, which may at a speed of 10,000 to 20,000 rpm; e) evaporating ethanol may comprise subjecting the CLCN solution to magnetic stirring for a period of time sufficient to produce a CLCNs essentially free of ethanol, such as for at least 10, 15, 20, 21, 22, 23, or 24 hours: f) the method may further comprise encapsulating RNA into the CLCNs; g) encapsulating may comprise adding an RNA solution to the CLCNs and vortexing to obtain RNA-loaded CLCNs; h) the RNA may be RNAi, such as siRNA or miRNA; i) the RNA may be added at a 1:1 volume ratio of CLCNs:RNA: j) at least 75%, such as at least 80%, 90%, 95%, or 100%, of the RNA may be encapsulated into the CLCNs; k) the method may not comprise chloroform, the formation of lipophilic film, or sonication.

Further embodiments provide cationic liquid crystalline nanoparticles (CLCNs) produced by a method as described above, including all of combinations of features described.

Additional embodiments provide methods of delivering RNA into a cell comprising administering an effective amount of RNA-loaded CLCNs as described above to a cell. The cell may be a human cell, a cancer cell, and/or an immune cell, such as a T cell.

In another embodiment, there are provided methods of treating a disease or disorder in a subject in need thereof comprising administering an effective amount of CLCNs described above, including all combinations of features described above.

The treatment method may have one or more of the following additional features, which may be combined with one another unless clearly mutually exclusive: a) the CLCNs may be loaded with RNA, such as RNAi, particularly siRNA or miRNA, such as miRNA mimics or inhibitors; b) the disease or disorder may be cancer, such as lung cancer, an inflammatory disorder, or an immune-associated disorder; c) the CLCNs may be loaded with miR150 inhibitor: d) the subject may be a human: e) the CLCNs may be administered orally, topically, intravenously, intraperitoneally, intramuscularly, endoscopically, percutaneously, subcutaneously, regionally, or by direct injection; f) the method may further comprise administering at least a second therapeutic agent, such as an anti-cancer agent.

In further embodiments, there are provided methods of immunostimulating a subject comprising administering an effective amount of CLCNs, including all combination of features above, to the subject. The CLCNs may loaded with immune-modulatory RNA. The CLCNs may be delivered to T cells. The CLCNs may result in an altered cytokine profile.

The CLCN's, including all combinations of features described above may also all be used in combination with the methods, including all combinations of features, described above. Both the compositions and methods described above may also be used in combination with any features described in this specification, including individual features of examples.

Other objects, features and advantages of the present disclosure will become apparent from the following detailed description. It may be understood, however, that the detailed description and the specific examples, while indicating preferred embodiments of the present disclosure, are given by way of illustration, since various changes and modifications within the spirit and scope of the present disclosure will become apparent to those skilled in the art from this detailed description.

### BRIEF DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIGS. 1A-1B: CLCNs of the embodiments. A. Schematic representation of CLCNs-RNAi binding. B. Transmission Electron Microscopy (TEM) imaging of hexasomes, cubosomes, and CLCNs. CLCNs were prepared by using high-speed homogenization and assembled with synthetic siRNA or miRNA molecules in nuclease-free water to create CLCN/siRNA or miRNA complexes.

FIGS. 2A-2F: Physicochemical characterization of CLCNs and CLCNs/siRNA complexes. A. Transmission electron microscope images of CLCN1 and CLCN1 after conjugation with siRNA. B. Transmission electron microscope images of CLCN2 and CLCN2 after conjugation with siRNA. C. Physicochemical characterization (size of CLCNs and CLCNs/siRNA, zeta potential of CLCNs and CLCNs/siRNA complex and polydispersity index [PDI] by dynamic light scattering. Amount of siRNA conjugated by fluorescence analysis (%). Size: (**) p value 0.0063; zeta potential: (**) p value 0.0022 and (**) p value 0.0016 (unpaired two-tailed Student t test). D. Gel retardation assay to evaluate the nanoparticle retardation inside the gel and the siRNA condensation inside the CLCNs and relative density of the bands (fold change value). E. CLCN1 and CLCN1-siRNA complex size distribution per milliliters of solution using a NanoSight instrument for NTA. (Table 1). F. CLCN2 and CLCN2-siRNA complex size distribution per milliliters of solution using a NanoSight instrument for NTA. (Table 1).

FIGS. 3A-3E: Cellular uptake by flow cytometry analysis and fluorescence microscopy image analysis. A. Fluorescence microscopy images, flow cytometry intensity analysis, and fluorescence intensity quantification after 24 hours of treatment with CLCNs D275 and CLCNs conjugated with siRNA CySon H1299 cells. B. Fluorescence microscopy images after 24 hours of treatment with CLCN1 D275 and CLCN1 conjugated with siRNA Cy5, ER, and CLCN1 D275/siRNA Cy5 complex and nucleus on H1299 cells. C. Fluorescence microscopy images after 24 hours of treatment with CLCN2 D275 and CLCN2 conjugated with siRNA Cy5, ER, and CLCN2 D275/siRNA Cy5 complex and nucleus on H1299 cells. D. Subcellular localization of CLCN1-siRNA by confocal fluorescence imaging analysis. The H1299 cells were treated with fluorescence (D275) CLCN1, CLCN1/siRNA Cy5 complex (siRNA fluorescence [Cy5]), and nucleus (DAPI). E. Subcellular localization of CLCN2-siRNA by confocal fluorescence imaging analysis. The H1299 cells were treated with fluorescence (D275) CLCN2, CLCN2/siRNA Cy5 complex (siRNA fluorescence [Cy5]), and nucleus (DAPI).

FIGS. 4A-4B: Intracellular trafficking of CLCNs in H1299 cells by TEM. A. Cellular uptake and processes of CLCNs in H1299 cells by TEM analysis after 2, 4, 6, 8, 12, and 24 hours of treatment. B. Schematic representation of cellular uptake and internalization of CLCNs on the cell surface.

FIGS. 5A-5E: CLCNs RNAi mediated gene-silencing and gene-expression evaluation. A. Silencing of GFP expression in H1299 cells cotransfected with GFP plasmid vectors and CLCNs-siGFP nanoparticles for 24 hours. Flow cytometry analysis and fluorescence microscope images showed a dramatic reduction in GFP fluorescence when H1299 cells were transfected with anti-GFP siRNA complexed to the CLCNs formulations. (***) p value 0.0005: (****) p value <0.0001 (unpaired two-tailed Student t test). B. Graphical representation of the GFP fluorescence intensity percentage detected with flow cytometry analysis. (**) p value 0.0019: (*) p value 0.0203 (unpaired two-tailed Student t test). C. Silencing of GFP expression in H1299 cells cotransfected with GFP plasmid vectors and CLCN-siGFP nanoparticles for 24 hours by fluorescence microscopy images. DharmaFect was used to compare CLCN transfection efficiency. D. Calculation of GFP fluorescence intensity in the area of the images by ImageJ software (1.46r, http://imagej.nih.gov/ij). The fluorescence intensity percentage in the cells treated with only GFP plasmid was used as a control to compare the fluorescence intensity of the samples. E. Quantification of cellular uptake of CLCNs-miR30b complexes by qRT-PCR at various miR30b concentration (25, 50, and 100 nM) after 24 hours of transfection. 25 nM, CLCN1-miR30b vs DharmaFect-miR30b p value 0.0003 (***) and CLCN2miR30b vs DharmaFect-miR30b p value 0.0053 (**). 50 nM, CLCN1miR30b vs DharmaFect-miR30b p value 0.0004 (***) and CLCN2miR30b vs DharmaFect miR30b p value 0.0225 (*).

FIGS. 6A-6E: Biodistribution of CLCNs by systemic administration and effect on gene expression in NSCLC tumor-bearing mice. A. Images of fluorescence-labeled CLCNs in organs and tumors from mice 24 hours after tail vein injection. The tissue sections were collected after 24 hours of treatment with CLCNs D275 (10 mg/kg). The control group was not treated with CLCNs. B. Quantification of fluorescence intensity by fluorescence microscopy images analysis by ImageJ software (1.46r, http://imagej.nih.gov/ij). C. The fluorescence intensity of D275 encapsulated in the CLCNs was measured in various organs and tumors with use of flow cytometry analysis at a wavelength 460 nm excitation and 580 nm emission. D. Representation of flow cytometry analysis fluorescence intensity percentage for each single tissue. Brain (**) p value 0.0078; liver (*) p value 0.0121 and (*) p value 0.0234; kidney (**) p value 0.0031; spleen (*) p value 0.0133 and (*) p value 0.0426; lung (**) p value 0.0063 and (*) p value 0.0477; tumor (**) p value 0.0013 and (***) p value 0.0007 (unpaired two-tailed Student t test). E. Quantification with qPCR of miR30b expression in different mouse tissues. The tissues sections were collected 24 hours after treatment with CLCN D275/miR30 b complexes (1.5 mg/kg).

FIGS. 7A-7E: CLCNs Toxicity In Vitro and evaluation of damages in organs function after CLCNs In Vivo treatment. A. Fibroblasts derived from lung tissue (WI-38) treated for 24, 48, and 72 hours with various CLCN concentrations ranging from 0.01 to 100 PM. Cytotoxicity was evaluated with the XTT assay. CLCN1 at 24 hours: (***) p value 0.0013; (***) p value 0.0009 (**) p value 0.0013: (***) p value 0.0006. CLCN1 at 72 hours: (**) p value 0.0026; (**) p value 0.0020; (**) p value 0.0081. CLCN2 at 24 hours: (***) p value 0.0003; (****) p value <0.0001; (****) p value <0.0001; (**) p value 0.0019. CLCN2 at 72 hours: (****) p value <0.0001: (**) p value 0.0060; (***) p value 0.0002; (**) p value 0.001 (unpaired two-tailed Student t test). B. Non-small cell lung cancer (H1299) treated for 24, 48, and 72 hours with various CLCN concentrations ranging from 0.01 to 100 μM. CLCN1 at 24 hours: (***) p value 0.0004; (***) p value 0.0004 (unpaired two-tailed Student t test). C. Non-small cell lung cancer (H1299) treated for 24, 48, and 72 hours with various CLCN-siRNA concentrations at 100 nM, 50 nM, and 100 μM. CLCN2-siGFP at 24 hours: (***) p value 0006: (**) p value 0.0024; (****) p value <0.0001. CLCN2-siGFP at 72 hours: (***) p value 0.0004; (**) p value 0.0027 (unpaired two-tailed Student t test). D. Biochemical values of mice blood 24 hours after CLCNs D275 systemic injection by tail vein. E. Routine histopathology analysis, H&E staining of major organs after 24 hour of CLCNs injection at 10 mg/Kg dose.

FIG. 8: Quantification of human T cells transfected with CLCNsD275 and CLCNsD275-miR124 nanoparticles by flow Cytometry. The mean fluorescence intensity of the Human T-cells transfected with CLCNsD275 or CLCNs-D275-miR124 was compared to that of the untreated group 24 hours after transfection. The statistical significance (p<0.0001) between treated and untreated groups were calculated by two-tailed student t test.

FIG. 9: Quantification of human T cells transfected with CLCNsD275 and CLCNsD275-miR124 nanoparticles by flow Cytometry. The mean fluorescence intensity of the Human T-cells transfected with CLCNsD275 or CLCNs-D275-miR124 was compared to that of the untreated group 48 hours after transfection. The statistical significance (p<0.0001) between treated and untreated groups were calculated by two-tailed student t test.

FIG. 10: Expression of miR-124 in human T-cells transfected by CLCNs-miR124 nanoparticles for 24 hours relative to that of untransfected cells. The expression of miR124 in the treated group was more than three-fold compared to that of untreated group (P value <0.0001).

FIG. 11: Knockdown of miR-150 expression in human T-cells after transfection using CLCNs-miR150 inhibitor (miR-150i) in vitro. The miR-150 expression was dramatically knocked down compared to the untreated group (P value 0.0002).

FIG. 12: Evaluation of Cytotoxicity of CLCNs and CLCNs-miR124 and CLCNs-miR-150i inhibitor on human T-cells, 24 and 48 hours after treatment at different concentrations of CLCNs (200 μM, 100 μM and 50 μM). CLCNs did not show any significant toxicity on human T-cells also at the highest concentration (200 μM).

FIG. 13: Expression of miR-124 after i.v injection of CLCNs-miR124 in C57BL/6 mice for 24 and 48 Hours. CLCNs were able to efficiently deliver synthetic miR-124 after 24 hours in vivo, as indicated by the expression fold changes in the treated group compared to the untreated group (NT) (P value <0.0030).

FIG. 14: Cytokines Expression in T-cells after treatment with CLCNs-miR124 in vivo for 24 and 48 hours. The selected cytokines and the co-stimulatory factors showed an increase in the relative expression after 48 hours of treatment with CLCNs-miR124.

### DESCRIPTION OF ILLUSTRATIVE EMBODIMENTS

In some embodiments, the present disclosure provides cationic liquid crystalline nanoparticles (CLCNs) which may be used an advanced delivery system, such as for delivering siRNA or miRNA mimics in vitro and in vivo to either induce gene silencing or increase gene expression upon transfection. The CLCNs provided herein display several advantages over current delivery systems including small size (e.g., less than 100 nm), decreased toxicity, longer half-life in circulation, and prolonged delivery over time. CLCNs can also minimize nonspecific opsonization, phagocytosis, and immune activation and promote interaction with the cellular surface. The homogeneous and stable CLCNs and CLCN-siRNA complexes may be about 100 nm in diameter, with positively charged surfaces. Indeed, the positive charge of the CLCNs enhances delivery of the nanoparticles to target cells as well as their internalization. The CLCNs are nontoxic (e.g., measured by the effect of the CLCNs on cell viability) and are taken up by human cells though endocytosis to deliver the RNAi to the cytoplasm.

Specifically, the present studies with the CLCN-siRNA complexes showed significant inhibition of gene expression detected in transiently transfected lung cancer H1299 cells treated with CLCNs/anti-GFP complexes 24 hours after transfection. Interestingly, biodistribution analysis showed that the CLCNs and CLCNs-RNAi complexes were successfully delivered to various organs and into the subcutaneous human lung cancer H1299 tumor xenografts in mice 24 hours after systemic administration. Thus, the CLCNs effectively deliver RNAi in vivo. These results suggest that CLCNs are a unique and advanced delivery system capable of protecting RNAi from degradation and of efficiently delivering RNAi in vitro and in vivo.

Furthermore, the present disclosure provides a fabrication method for the CLCNs which is an efficient and cost-effective process for producing CLCNs, such as for use as RNAi delivery systems. Specifically, the method for producing the CLCNs may comprise high-speed homogenization of a cationic phospholipid and glycerol monooleate with a nonionic surfactant. The CLCNs may then be successfully conjugated with siRNA or miRNA based on electrostatic interaction with the cationic lipid, such as 1,2-dioleoyl-3-trimethylammonium-propane (chloride salt) (DOTAP). The results of the present studies showed that a low concentration of DOTAP, such as less than 20%, and nonionic surfactant, such as less than 1%, can induce efficient binding between the carrier and the RNAi and reduce toxicity in vitro and in vivo. Thus, the CLCNs developed in this study offer an alternative approach for delivering siRNA or miRNA with the advantages of being prepared from physiologically well-tolerated materials and of having an efficient delivery system to silence or activate gene expression in vitro and in vivo.

In addition, the CLCNs of the present disclosure were shown to have uptake and internalization by immune cells, particularly T cells. The CLCN-miRNA complexes were not toxic to the T cells and were observed to silence gene expression as well as induce changes in cytokine secretion by the T cells. Thus, the CLCNs of the present disclosure may also be used as delivery systems to immune cells, such as for immunostimulation.

### I. DEFINITIONS

As used herein, “essentially free,” in terms of a specified component, is used herein to mean that the specified components have not been purposefully formulated into a composition and/or is present as a contaminant or in trace amounts. The total amount of the specified component resulting from any unintended contamination of a composition is therefore well below 0.05%. Preferably is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives or the alternatives are mutually exclusive, although the disclosure supports a definition that refers to alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

As used herein, the term “nanoparticle” refers to particles of any shape having at least one dimension that is less than about 150 nm.

“Crystalline nanoparticles” refer to those nanoparticles that have a substantially uniform, repeating three-dimensional structure. The terms “nanoparticles,” “crystalline nanoparticles,” and “cationic liquid crystalline nanoparticles (CLCNs)” may be used interchangeably herein to refer to the nanoparticles of the present disclosure.

“Treatment” and “treating” as used herein refer to administration or application of a therapeutic agent to a subject or performance of a procedure or modality on a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, reduction in the size of a tumor.

“Subject” and “patient” refer to either a human or non-human, such as primates, mammals, and vertebrates. In particular embodiments, the subject is a human.

The term contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

A “therapeutic agent” as used herein refers to any agent that can be administered to a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

A “diagnostic agent” as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA. In particular embodiments, the therapeutic or diagnostic agent is a siRNA.

A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between 3 and about 100 nucleobases in length. The term “polynucleotide” refers to at least one molecule of greater than about 100 nucleobases in length.

These definitions refer to a single-stranded or double-stranded nucleic acid molecule. Double stranded nucleic acids are formed by fully complementary binding, although in some embodiments a double stranded nucleic acid may formed by partial or substantial complementary binding. Thus, a nucleic acid may encompass a double-stranded molecule that comprises one or more complementary strand(s) or “complement(s)” of a particular sequence, typically comprising a molecule. As used herein, a single stranded nucleic acid may be denoted by the prefix “ss” and a double stranded nucleic acid by the prefix “ds”.

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art.

The term “siRNA” (short interfering RNA) refers to short double stranded RNA complex, typically 19-28 base pairs in length. In other words, siRNA is a double-stranded nucleic acid molecule comprising two nucleotide strands, each strand having about 19 to about 28 nucleotides (i.e., about 19, 20, 21, 22, 23, 24, 25, 26, 27, or 28 nucleotides). The complex often includes a 3′-overhang. siRNA can be made using techniques known to one skilled in the art and a wide variety of siRNA is commercially available from suppliers such as Integrated DNA Technologies, Inc. (Coralville, Iowa). In one embodiment, a 2′-O-methyl-modified siRNA duplex against TNF-α as described herein can be incorporated into the nanoparticles, wherein the 2′-O-methyl modification on the anti-sense strand eliminates off-target effects, minimizes nonspecific immune responses, and improves siRNA stability.

A “microRNA (miRNA)” is short, non-coding RNAs that can target and substantially silence protein coding genes through 3′-UTR elements. miRNAs can be approximately 21-22 nucleotides in length and arise from longer precursors, which are transcribed from non-protein-encoding genes.

An “immune disorder,” “immune-related disorder.” or “immune-mediated disorder” refers to a disorder in which the immune response plays a role in the development or progression of the disease. Immune-mediated disorders include autoimmune disorders, allograft rejection, graft versus host disease and inflammatory and allergic conditions.

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

As used herein, “Pluronic F-127” refers to a compound of CAS No. 9003-11-6.

### II. CATIONIC LIQUID CRYSTALLINE NANOPARTICLES (CLCNs)

Certain embodiments of the present disclosure concern CLCNs and methods of their production. The CLCNs may be comprised of a mixture of one or more cationic phospholipids, glycerol monooleate (GMO) (e.g., 1-(cis-9-octadecenoyl)-rac-glycerol), and a nonionic surfactant.

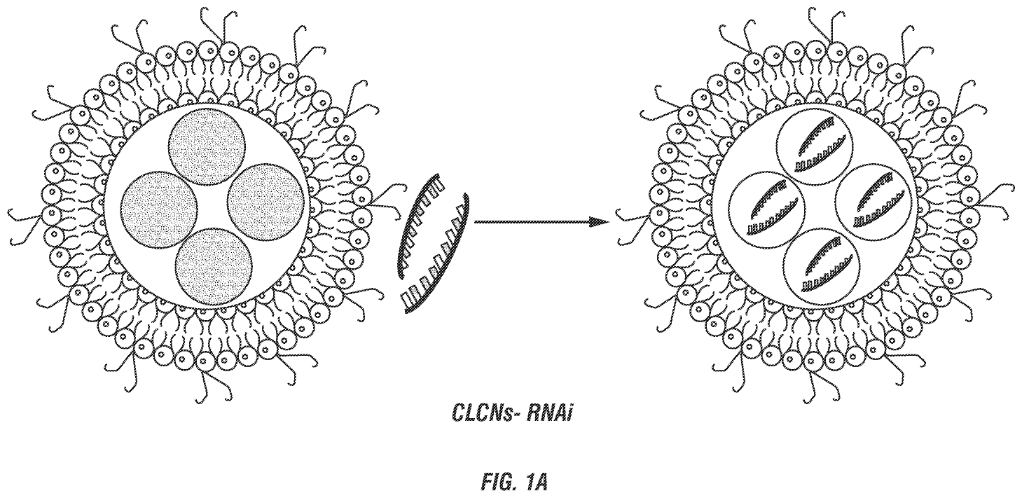

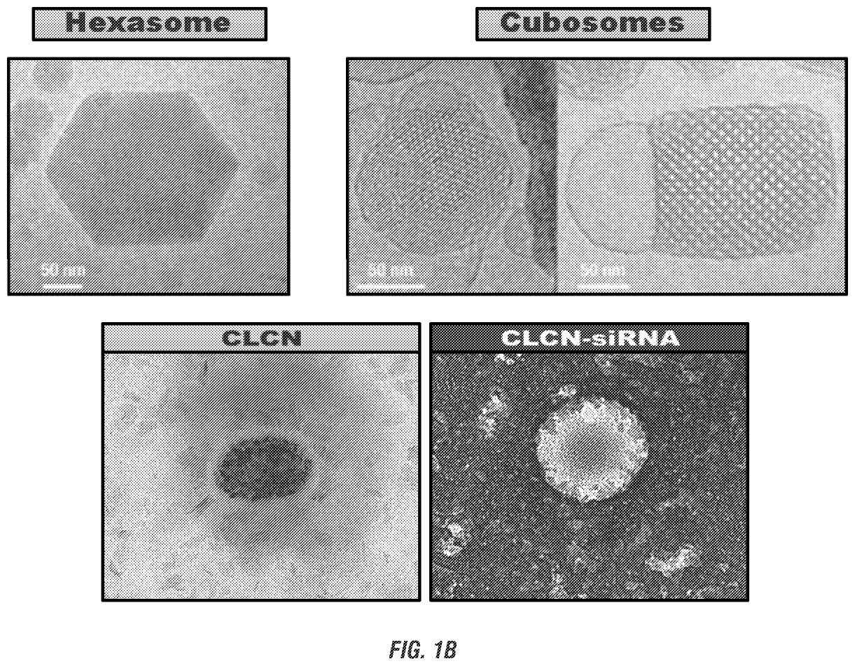

In particular aspects, the CLCNs of the present disclosure are circular phospholipid bilayer nanoparticles with a hydrophobic shell and aqueous core that have a liquid crystalline phase in solution. These colloidal nanoparticles are distinct from hexasomes or cubosomes as seen by TEM images in FIG. 1B due to the method of producing the present nanoparticles.

The CLCNs are small and preferably comprise a diameter less than 120 nm, particularly less than 100 nm. The diameter of the CLCNs may be about 50-150 nm, such as 60-100 nm. Specifically, the diameter may be about 50, 55, 60, 65, 70, 75, 80, 85, 90 or 95 nm.

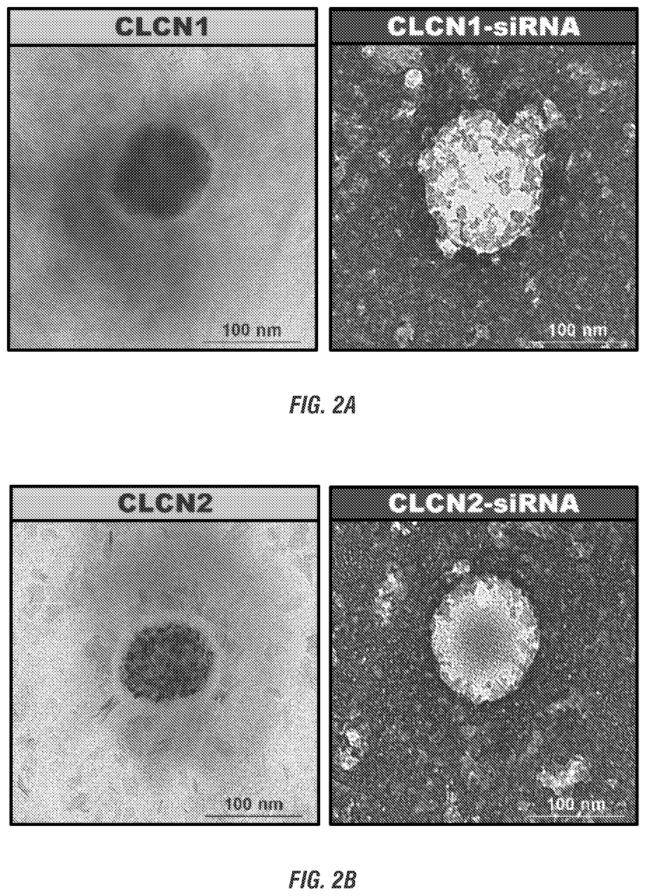

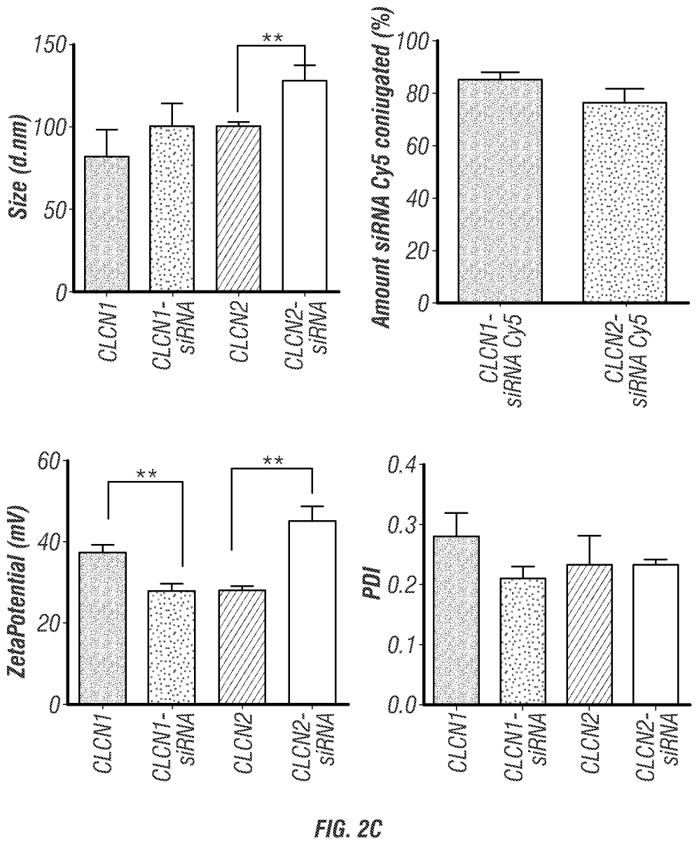

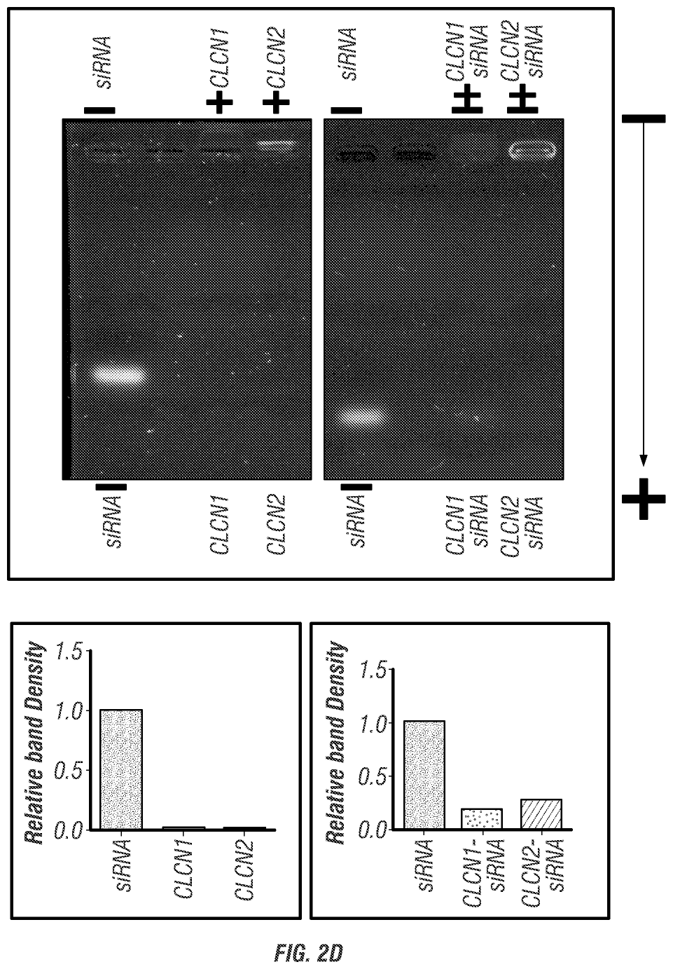

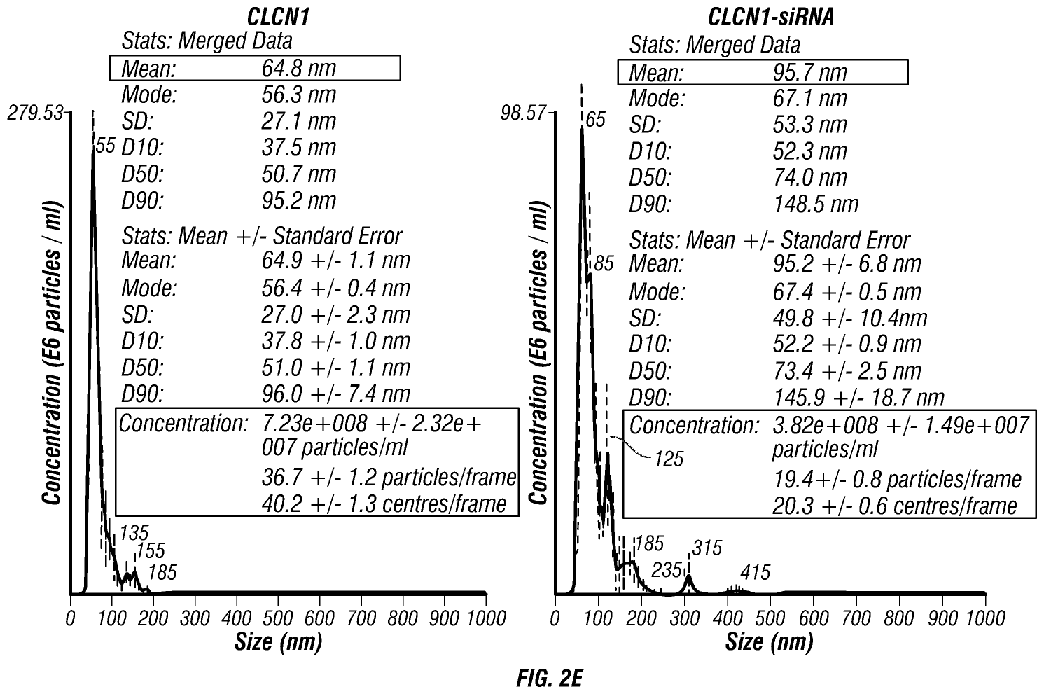

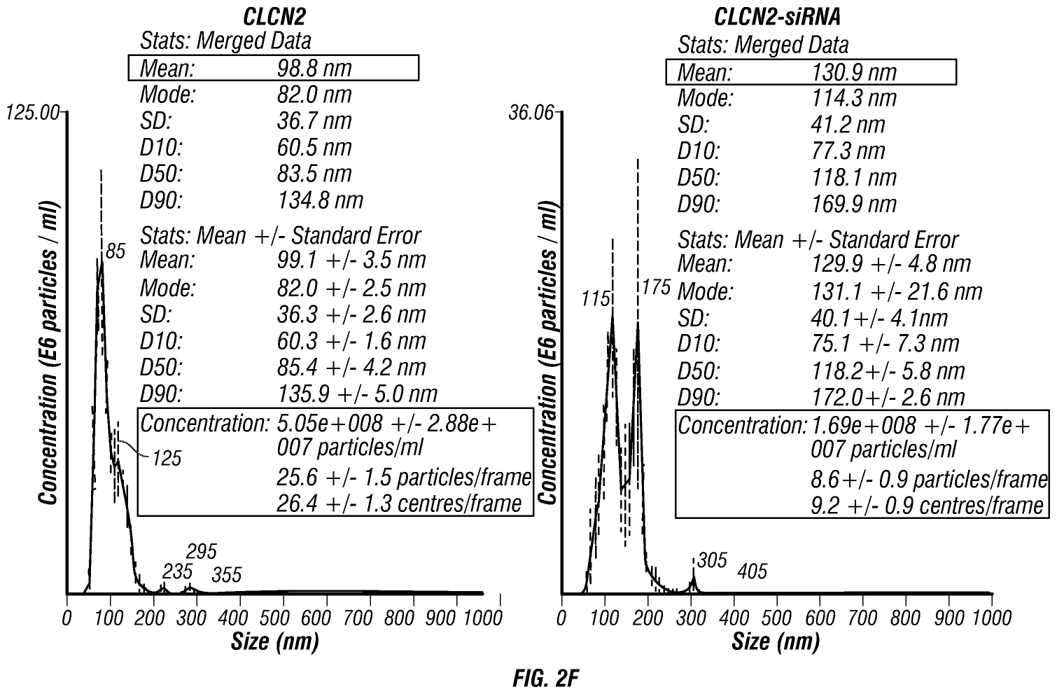

The unloaded CLCNs (i.e., not comprising a therapeutic agent or diagnostic agent) are positively charged (see FIG. 2C), such as with a zeta potential greater than +20 mV, particularly greater than +30 mV, such as +31, +32, +33, +34, +35, +36, +37, +38, +39, or +40 mV. Specifically, the zeta potential may be between +25 and +35 mV, such as +20 to +40, +30 to +50, or +35 to +45 mV. Generally, the zeta potential increases when the CLCNs are loaded with negatively-charged RNA, such as siRNA or miRNA mimics, as the RNA is encapsulated in the aqueous core of the CLCNs and surrounded by the lipid bilayer and hydrophobic shell. For example, the zeta potential of CLCN-siRNA or CLCN-miRNA complexes may be increased by at least 2 mV, such as at least 5, 6, 7, 8, 9, or 10 mV as compared to unloaded CLCNs. A CLCN-siRNA complex may have a zeta potential between +30 and +45 mV, such as at least +35, +40, +45, +50, or +55 mV.

In particular embodiments, the CLCNs are homogenous and stable nanoparticles, such as demonstrated by a low polydispersity index (PDI). The PDI may be less than 0.3, such as less than 0.2, particularly less than 0.15. For example, the PDI may be between 0.01 to 0.30, such as 0.05 to 0.25, particularly 0.10 to 0.20.

The one or more cationic phospholipids in the CLCNs may be 2-dioleoyl-3-trimethylammonium-propane chloride salt (DOTAP), Dimethyldioctadecylammonium (DDAB), 1,2-di-O-octadecenyl-3-trimethylammonium propane (DOTMA), 3ß-[N—(N′,N′-dimethylaminoethane)-carbamoyl]cholesterol hydrochloride (DC-CHOL), 1,2-dioleoyl-3-dimethylammonium-propane (DODAP), cetyl trimethyl ammonium bromide (CTAB), 1,3-dioleoyloxy-2-(6-carboxy-spermyl)-propyl-amide (DOSPER), and 1,2-dioleoyl-sn-glycero-3-[(N-(5-amino-1-carboxypentyl)iminodiacetic acid)succinyl](DOGS). The CLCNs may comprise 1, 2, 3, 4, or more cationic phospholipids. In one embodiment, the CLCNs comprise one cationic phospholipid, such as DOTAP.

In particular embodiments, the CLCNs comprise one cationic phospholipid, particularly DOTAP, at a low concentration, such as less than 30%, particularly less than 25%, more particularly less than 20%. The DOTAP may be comprised in the CLCNs at a weight percentage of about 5-25%, such as 6-20%, 5-10%, 6-12%, 7-15%, 9-16%, 10-19%, or 12-20%. In particular aspects, the DOTAP is comprised at a weight percent of about 7%, 7.5%, 15%, or 18%.

Examples of nonionic surfactants for use in the presently disclosed methods and compositions include polysorbates, including but not limited to, polyethoxylated sorbitan fatty acid esters (e.g., TWEEN® compounds) and sorbitan derivatives (e.g., SPAN® compounds); ethylene oxide/propylene oxide copolymers (e.g., PLURONIC® compounds, which are also known as poloxamers); polyoxyethylene ether compounds, such as those of the BRIJ®® family, including but not limited to polyoxyethylene stearyl ether (also known as polyoxyethylene (100) stearyl ether and by the trade name BRIJ® 700); and ethers of fatty alcohols. In particular embodiments, the nonionic surfactant is a nonionic polyol, such as Pluronic F-127. In particular aspects, the nonionic surfactant is present in the CLCNs at a low concentration, such as less than 10%, particularly less than 9, 8, 7, 6, 5, 4, 3, 2, or 1% by weight. Specifically, the nonionic surfactant (e.g., Pluronic F127) is present at a concentration less than 1%, such as 0.9, 0.8, 0.7, 0.6, 0.5, 0.4, 0.3, 0.2, or 0.1% by weight, particularly about 0.5%.

The CLCNs may further comprise one or more additional components, such as for increased stability or efficiency in delivery. For example, the CLCNs may comprise one or more neutral phospholipids, such as dioleoylphosphatidylethanolamine (DOPE), phospholipid 1,2-diphytanoyl-sn-glycero-3-phosphoethanolamine (DPhPE), diolcoyl-phosphatidylcholine (DOPC), 1,2-dihexadecylphosphoethanolamine, 1,2-dilauroylphosphoethanolamine, 1,2-dilinoleoylphosphoethanol-amine, 1,2-dimyristoylphosphoethanolamine, 1,2-dioleoylphosphor-ethanolamine, 1,2-dipalmitoylphosphoethanolamine, 1,2-distearoylphosphoethanolamine, 1-palmitoyl-2-oleoylphospho-ethanolamine (POPE), 1,2-dipalmitoylphosphoethanolamine-N-[4 (p-maleimidephenyl) butyla-mide], 1,2-dipalmitoylphosphocthanol-amine-N-[3-(2-pyridyldithio) propionate], 1,2-dioleoylphospho-ethanolamine-N-(succinyl) and 1,2-dipalmitoylphosphoethanol-amine-N-(succinyl). The CLCNs may comprise one or more ionizable cationic lipids such as 1,2-DiLinoleyloxy-N,N-dimethylaminopropane (DLinDMA), 1,2-dilinoleyl-4-(2-dimethylaminoethyl)[1,3]-dioxolane (DLinKC2-DMA), dilinoleyl-methyl-4-dimethylaminobutyrate (DLin-MC3-DMA), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoylolcoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl phosphatidyl ethanolamine, 16-O-dimethyl phosphatidyl ethanolamine, 18-1-trans phosphatidyl ethanolamine, 1-stearoyl-2-oleoyl-phosphatidyethanolamine (SOPE), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethvlammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA), and 1,2-Dilinolenyloxy-N,N-dimethylaminopropane (DLendMA).

A. Production of CLCNs

The present disclosure further provides methods for producing CLCNs which are extremely efficient and cost-effective. As compared to standard liposomal production methods, in particular aspects, the present methods do not comprise the formation of a lipophilic film (e.g., use of a Rotovapor) or a sonication step in order to produce the small, stable, and homogenous CLCNs provided herein. In addition, in particular aspects, solubilization of the CLCN components does not comprise the use of organic compounds, such as chloroform. Instead, the present methods use an alcohol, such as ethanol, and water as solvents. The present methods may comprise converting two immiscible liquids into an emulsion using high-speed homogenization and keeping the emulsion stable using a low weight percentage of nonionic surfactant, such as Pluronic F127.

Generally, the present methods for producing the nanoparticles can comprise solubilizing the one or more cationic phospholipids and glycerol monooleate in an alcohol solvent, such as ethanol or methanol, particularly ethanol, to produce a lipophilic phase. The nonionic surfactant is solubilized in water, such as RNAse-free water, to produce a hydrophilic phase. The lipophilic phase is then subjected to high-speed homogenization while the hydrophilic phase is added dropwise to the lipophilic phase. The solution may then be subjected to one or more rounds, such as 2, 3, 4, or 5 rounds, of high-speed homogenization which may be at a speed greater than the homogenization speed during mixing. The alcohol solvent is then evaporated for a period of time sufficient to produce a CLCN solution essentially free of alcohol, such as at least 10 hours, particularly 15, 20, 21, 22, 23, or 24 hours. It may be preferable to perform the CLCN production at a temperature less than room temperature, such as less than 10° C., particularly around 4° C.

The molar ratio of the glycerol monooleate to cationic phospholipid, such as GMO:DOTAP (e.g., calculated for 50 mg of lipids in 5 mL of solution), may be about 40-49:10-1, such as 40:10, 41:9, 42:8, 43:7, 44:6, 45:5, 46:4, 47:3, 48:2, or 49:1. The weight percentage of the cationic lipid, such as DOTAP, in the CLCNs may be about 2-30%, such as 5-20%, specifically 6-19%, such as 7, 8, 9, 10, 11, 12, 13, 14, 15, 16, 17, 18, or 19% cationic lipid.

High-speed homogenization may be at a speed of about 5,000 to 30.000 rpm, such as 6,000 to 10,000 rpm, 7,000 to 12,000 rpm, 9,000 rpm to 15,000 rpm, 10,000 rpm to 20,0000 rpm, or 25,000 to 30,000 rpm. The CLCNs may be homogenized at different speeds throughout the production process, such as about 5,000-10,000 rpm in a first step of homogenization and about 10,000 to 20,000 rpm in a second step of homogenization. High-speed homogenizers that may be used for the present methods include, but are not limited to, high speed rotor/stator homogenizers such as those commercially available from Nition, Kinematica, Hitachi, Homogenizer Polytron, or IKA Ultra-Turrax, particularly the IKAX ULTRA-TURRAX-25.

In one exemplary method, the CLCNs are produced by solubilizing 1-(cis-9-Octadecenoyl)-rac-glycerol (GMO) and 2-dioleoyl-3-trimethylammonium-propane (chloride salt) (DOTAP) in ethanol. The Pluronic F127 is solubilized at 4° C. in RNAsi-free water. The polymeric solution of the Pluronic F27 is added drop-by-drop to the lipophilic mixture of GMO/DOTAP under high speed homogenization at 8000-9000 rpm (IKAULTRA-TURRAXT-25). The solution is then homogenized at a higher speed of 14,000 rpm for about 5 minutes three times with a break of 5 minutes between each homogenization. The solution is then placed on a magnetic stirring plate for about 24 hours for ethanol evaporation. The obtained CLCNs may be stored at 4° C.

B. Loading of CLCNs

The CLCNs of the present disclosure may be loaded with a therapeutic agent or diagnostic agent for use as a delivery vehicle. The therapeutic agent may be RNA, such as siRNA, shRNA, plasmid, mRNA, miRNA, or ncRNA, particularly siRNA or miRNA therapeutics. The miRNA may be a miRNA mimic, or a miRNA precursor. The size of the RNA loaded into the CLCNs may be less than 100 nucleotides in length, such as less than 75 nucleotides, particularly less than 50 nucleotides in length. For example, the RNA may have a length of about 10-100 nucleotides, such as 20-50 nucleotides, particularly 10-20, 15-25, 20-30, 25-35, 30-40, or 45-50 nucleotides.

The CLCNs may be loaded with the therapeutic agent, such as siRNA or miRNA mimic, by vortexing. For example, the CLCNs and RNA may be mixed at a 1:1 ratio and vortexed for about 1 minute to encapsulate the RNA intro the CLCNs. The ratio of CLCN to RNA may be 1:1, 1:2, 1:3, 1:4, 2:1, 3:1, 1:1.1, 1:1.2, 1:1.3, 1:1.4, or 1:1.5.

The RNA may be modified or non-modified. The RNA may comprise an alteration of one or more nucleotides. Such alterations can include the addition of non-nucleotide material, such as to the end(s) of the RNA or internally (at one or more nucleotides of the RNA). In certain aspects, the RNA molecule contains a 3′-hydroxyl group. Nucleotides in the RNA molecules of the present disclosure can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Publication No. 20040019001 and U.S. Pat. No. 6,673,611 (each of which is incorporated by reference in its entirety). Collectively, all such altered nucleic acids or RNAs described above are referred to as modified siRNAs.

Preferably, RNAi is capable of decreasing the expression of a protein by at least 10%, 20%, 30%, or 40%, more preferably by at least 50%, 60%, or 70%, and even more preferably by at least 75%, 80%, 90%, 95% or more.

The siRNA as used in the methods or compositions described herein may comprise a portion which is complementary to an mRNA sequence encoded by NCBI Reference Sequence for the stated genes/proteins. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. In an embodiment, the overhang is UU. The siRNA can be 5′ phosphorylated or not and may be modified with any of the known modifications in the art to improve efficacy and/or resistance to nuclease degradation. In a non-limiting embodiment, the siRNA can be administered such that it is transfected into one or more cells. In one embodiment, a siRNA may comprise a double-stranded RNA comprising a first and second strand, wherein one strand of the RNA is 80, 85, 90, 95 or 100% complementary to a portion of an RNA transcript of a gene.

In one embodiment, a single strand component of a siRNA of the present disclosure is from 14 to 50 nucleotides in length. In another embodiment, a single strand component of a siRNA is 14, 15, 16, 17, 18, 19, 20, 21, 22, 23, 24, 25, 26, 27, or 28 nucleotides in length. In yet another embodiment, a single strand component of a siRNA of the present disclosure is 21 nucleotides in length. In yet another embodiment, a single strand component of a siRNA of the present disclosure is 22 nucleotides in length. In yet another embodiment, a single strand component of a siRNA of the present disclosure is 23 nucleotides in length. In one embodiment, a siRNA of the present disclosure is from 28 to 56 nucleotides in length.

A target gene generally means a polynucleotide comprising a region that encodes a polypeptide, or a polynucleotide region that regulates replication, transcription or translation or other processes important to expression of the polypeptide, or a polynucleotide comprising both a region that encodes a polypeptide and a region operably linked thereto that regulates expression. The targeted gene can be chromosomal (genomic) or extrachromosomal. It may be endogenous to the cell, or it may be a foreign gene (a transgene). The foreign gene can be integrated into the host genome, or it may be present on an extrachromosomal genetic construct such as a plasmid or a cosmid. The targeted gene can also be derived from a pathogen, such as a virus, bacterium, fungus or protozoan, which is capable of infecting an organism or cell. Target genes may be viral and pro-viral genes that do not elicit the interferon response, such as retroviral genes. The target gene may be a protein-coding gene or a non-protein coding gene, such as a gene which codes for ribosomal RNAs, splicosomal RNA, tRNAs, etc.

Any gene being expressed in a cell can be targeted. Preferably, a target gene is one involved in or associated with the progression of cellular activities important to disease or of particular interest as a research object. Thus, by way of example, the following are classes of possible target genes that may be used in the methods of the present disclosure to modulate or attenuate target gene expression: developmental genes (e.g., adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), tumor suppressor genes (e.g., APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WTI, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide), pro-apoptotic genes (e.g., CD95, caspase-3, Bax, Bag-1, CRADD, TSSC3, bax, hid, Bak, MKP-7, PARP, bad, bcl-2, MST1, bbc3, Sax, BIK, and BID), cytokines (e.g., GM-CSF, G-CSF, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-1ß, TGF-β, TNF-α, TNF-β, PDGF, and mda7), oncogenes (e.g., ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES), and enzymes (e.g., ACP desaturases and hycroxylases, ADP-glucose pyrophorylases, ATPases, alcohol dehycrogenases, amylases, amyloglucosidases, catalases, cellulases, cyclooxygenases, decarboxylases, dextrinases, esterases. DNA and RNA polymerases, galactosidases, glucanases, glucose oxidases. GTPases, helicases, hemicellulases, integrases, invertases, isomersases, kinases, lactases, lipases, lipoxygenases, lysozymes, pectinesterases, peroxidases, phosphatases, phospholipases, phophorylases, polygalacturonases, proteinases and peptideases, pullanases, recombinases, reverse transcriptases, topoisomerases, xylanases).

In some embodiments, the CLCNs may be loaded with analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antiasthamatics (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline); antidiabetics (e.g., biguanides and sulfonylurea derivatives), antifungal agents (e.g., griseofulvin, ketoconazole, itraconizole, amphotericin B, nystatin, and candicidin); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone); antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, propanolol, isometheptene mucate, and dichloralphenazone); sedatives/hypnotics (e.g., barbiturates such as pentobarbital, pentobarbital, and secobarbital, and benzodiazapines such as flurazepam hydrochloride, triazolam, and midazolam); antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine); antimanic agents (e.g., lithium carbonate); antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium); antigout agents (e.g., colchicine, and allopurinol); anticoagulants (e.g., heparin, heparin sodium, and warfarin sodium): thrombolytic agents (e.g., urokinase, streptokinase, and alteplase); antifibrinolytic agents (e.g., aminocaproic acid); hemorheologic agents (e.g., pentoxifylline); antiplatelet agents (e.g., aspirin); anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine, and): agents useful for calcium regulation (e.g., calcitonin, and parathyroid hormone); antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, ccphradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium: penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium: erythromycins such as erythromycin ethylsuccinate, erythromycin, erythromycin estolate, erythromycin lactobionate, erythromycin stearate, and erythromycin ethylsuccinate: and tetracyclines such as tetracycline hydrochloride, doxycycline hyclate, and minocycline hydrochloride, azithromycin, clarithromycin); anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate: anticholinergic agents such as ipratropium bromide: xanthines such as aminophylline, dyphylline, metaproterenol sulfate, and aminophylline; mast cell stabilizers such as cromolyn sodium: inhalant corticosteroids such as beclomethasone dipropionate (BDP), and beclomethasone dipropionate monohydrate; salbutamol: ipratropium bromide; budesonide; ketotifen: salmeterol; xinafoate; terbutaline sulfate; triamcinolone: theophylline: nedocromil sodium; metaproterenol sulfate; albuterol; flunisolide: fluticasone proprionate: steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate: corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cvpionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, glyburide, chlorpropamide, glipizide, tolbutamide, and tolazamide); hypolipidemic agents (e.g., clofibrate, dextrothyroxine sodium, probucol, pravastitin, atorvastatin, lovastatin, and niacin); proteins (e.g., DNase, alginase, superoxide dismutase, and lipase): nucleic acids (e.g., sense or anti-sense nucleic acids encoding any therapeutically useful protein, including any of the proteins described herein); agents useful for erythropoiesis stimulation (e.g., erythropoietin): antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); oil-soluble vitamins (e.g., vitamins A, D, E, K, and the like): as well as other drugs such as mitotane, halonitrosoureas, anthrocyclines, and ellipticine.

### III. METHODS OF USE

In some embodiments, the present disclosure provides methods of using the CLCNs provided herein for the delivery of a therapeutic agent, such as RNAi, to a cell. The cell may be in vivo or ex vivo. In one embodiment, there is provided a method of delivering RNA into a cell comprising administering an effective amount of CLCN encapsulating RNAi to the cell. The cell may be a T cell. In a further embodiment, there is provided a method of immunostimulating an organism comprising administering an effective amount of CLCNs encapsulating RNA to the subject. The RNA may be an immune-modulatory RNA. In another embodiment, there is provided a method of treating a subject with a disease or disorder comprising administering an effective amount of the CLCNs of the present disclosure. In some embodiments, there is provided the use of the CLCNs of the present disclosure for the treatment of a disease or disorder or for immunostimulating a subject.

The m vivo cell can be in any subject, such as a mammal. For example, the subject may be a human, a mouse, a rat, a rabbit, a dog, a cat, a cow, a horse, a pig, a goat, a sheep, a primate, or an avian species. In particular embodiments, the subject is a human. For example, the human may be a subject with a disease. The disease may be any disease that afflicts a subject, such as an inflammatory disease, a hyperproliferative disease, an infectious disease, or a degenerative disease. In particular embodiments, the disease is a hyperproliferative disease such as cancer. For example, the cancer may be breast cancer, lung cancer, prostate cancer, ovarian cancer, brain cancer cell, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, intestinal cancer, lymphoma, or leukemia. In particular embodiments, the cancer is ovarian cancer.

Certain embodiments of the present disclosure concern methods of treating or preventing disease in a subject involving administration of CLCNs of the present disclosure. The disease may be any disease that can affect a subject. For example, the disease may be a hyperproliferative disease, an inflammatory disease, or an infectious disease. In particular embodiments, the disease is a hyperproliferative disease. In more particular embodiments, the disease is cancer.

The cancer may be a solid tumor, metastatic cancer, or non-metastatic cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer or breast cancer. In addition, the cancer may specifically be of the following histological type, though it is not limited to these: neoplasm, malignant; carcinoma: carcinoma, undifferentiated; giant and spindle cell carcinoma; small cell carcinoma; papillary carcinoma; squamous cell carcinoma: lymphoepithelial carcinoma: basal cell carcinoma: pilomatrix carcinoma: transitional cell carcinoma: papillary transitional cell carcinoma; adenocarcinoma; gastrinoma, malignant; cholangiocarcinoma: hepatocellular carcinoma: combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma: carcinoid tumor, malignant; branchiolo-alveolar adenocarcinoma papillary adenocarcinoma; chromophobe carcinoma; acidophil carcinoma: oxyphilic adenocarcinoma; basophil carcinoma; clear cell adenocarcinoma: granular cell carcinoma: follicular adenocarcinoma; papillary and follicular adenocarcinoma: nonencapsulating sclerosing carcinoma; adrenal cortical carcinoma: endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma: medullary carcinoma: lobular carcinoma; inflammatory carcinoma; paget's disease, mammary: acinar cell carcinoma: adenosquamous carcinoma; adenocarcinoma w/squamous metaplasia: thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant: paraganglioma, malignant; extra-mammary paraganglioma, malignant; pheochromocytoma; glomangiosarcoma; malignant melanoma: amelanotic melanoma; superficial spreading melanoma; malignant melanoma in giant pigmented nevus; epithelioid cell melanoma; blue nevus, malignant; sarcoma; fibrosarcoma fibrous histiocytoma, malignant: myxosarcoma: liposarcoma; leiomyosarcoma: rhabdomyosarcoma; embryonal rhabdomyosarcoma: alveolar rhabdomyosarcoma; stromal sarcoma; mixed tumor, malignant; mullerian mixed tumor; nephroblastoma; hepatoblastoma: carcinosarcoma: mesenchymoma, malignant: brenner tumor, malignant; phyllodes tumor, malignant: synovial sarcoma: mesothelioma, malignant; dysgerminoma; embryonal carcinoma: teratoma malignant; struma ovarii, malignant; choriocarcinoma; mesonephroma, malignant: hemangiosarcoma; hemangioendothcelioma, malignant: kaposi's sarcoma; hemangiopericytoma, malignant: lymphangiosarcoma: osteosarcoma; juxtacortical osteosarcoma; chondrosarcoma; chondroblastoma, malignant; mesenchymal chondrosarcoma; giant cell tumor of bone; ewing's sarcoma: odontogenic tumor, malignant; ameloblastic odontosarcoma: ameloblastoma, malignant: ameloblastic fibrosarcoma; pinealoma, malignant; chordoma: glioma, malignant: ependymoma: astrocytoma: protoplasmic astrocytoma; fibrillary astrocytoma; astroblastoma: glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma: neurilemmoma, malignant; granular cell tumor, malignant; malignant lymphoma: hodgkin's disease; hodgkin's: paragranuloma; malignant lymphoma, small lymphocytic; malignant lymphoma, large cell, diffuse; malignant lymphoma, follicular; mycosis fungoides: other specified non-hodgkin's lymphomas; malignant histiocytosis; multiple myeloma; mast cell sarcoma; immunoproliferative small intestinal disease; leukemia; lymphoid leukemia; plasma cell leukemia; crythroleukemia; lymphosarcoma cell leukemia; myeloid leukemia: basophilic leukemia; eosinophilic leukemia; monocytic leukemia; mast cell leukemia: megakaryoblastic leukemia; myeloid sarcoma; and hairy cell leukemia. Nonetheless, it is also recognized that the present disclosure may also be used to treat a non-cancerous disease (e.g., a fungal infection, a bacterial infection, a viral infection, and/or a neurodegenerative disease).

The RNA delivered by the CLCNs may be therapeutic or immunostimulatory, such as for use as a vaccine. In particular aspects, the CLCNs may deliver an immune-modulatory siRNA.

The CLCNs may be used to deliver RNA to immune cells, such as T cells. Additional immune cells that may be targeted by the CLCNs for delivery include dendritic cells, NK cells, and/or B cells.

In further embodiments, the therapeutic agent delivered by the CLCNs of the present disclosure may be a small molecule, vaccine, or an antigen.

In some embodiments, there is provided a method of treating a disease or disorder in a subject comprising administering an effective amount of CLCNs loaded with a therapeutic agent to a subject in need thereof. The disease may be an immune-associated disease, such as an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyncuropathy, Churg-Strauss syndrome, cicatrical pemphigoid. CREST syndrome, cold agglutinin disease, Crohn's disease, discoid lupus, essential mixed cryoglobulinemia, fibromyalgia-fibromyositis, glomerulonephritis. Graves' disease, Guillain-Barre. Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

A. Pharmaceutical Compositions

Certain of the methods set forth herein pertain to methods involving the administration of a pharmaceutically effective amount of a composition comprising CLCNs of the present disclosure.

1. Compositions

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated. The compositions used in the present disclosure may comprise different types of carriers depending on whether it is to be administered in solid, liquid or aerosol form, and whether it need to be sterile for such routes of administration as injection.

The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations preferably meet sterility, pyrogenicity, general safety and purity standards as required by FDA Office of Biologics standards.

The compositions comprising nanoparticles may be extensively dialyzed to remove undesired small molecular weight molecules and/or lyophilized for more ready formulation into a desired vehicle, where appropriate. The active compounds will then generally be formulated for administration by any known route, such as parenteral administration. Methods of administration are discussed in greater detail below.

The present disclosure contemplates methods using compositions that are sterile solutions for intravascular injection or for application by any other route as discussed in greater detail below. A person of ordinary skill in the art would be familiar with techniques for generating sterile solutions for injection or application by any other route. Sterile injectable solutions are prepared by incorporating the active compounds in the required amount in the appropriate solvent with various of the other ingredients familiar to a person of skill in the art.

The formulation of the composition may vary depending upon the route of administration. For parenteral administration in an aqueous solution, for example, the solution should be suitably buffered and the liquid diluent first rendered isotonic with sufficient saline or glucose. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, formulations for administration via an implantable drug delivery device, and any other form. One may also use nasal solutions or sprays, aerosols or inhalants in the present disclosure.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations.

In certain embodiments, pharmaceutical composition includes at least about 0.1% by weight of the active agent. The composition may include, for example, about 0.01%. In other embodiments, the pharmaceutical composition includes about 2% to about 75% of the weight of the composition, or between about 25% to about 60% by weight of the composition, for example, and any range derivable therein.

The pharmaceutical composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition may be be stable under the conditions of manufacture and storage, and preserved against the contaminating action of microorganisms, such as bacteria and fungi. It will be appreciated that exotoxin contamination should be kept minimally at a safe level, for example, less than 0.5 ng/mg protein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride or combinations thereof.

In other embodiments, one may use nasal solutions or sprays, aerosols or inhalants in the present disclosure. Nasal solutions may be aqueous solutions designed to be administered to the nasal passages in drops or sprays.

Sterile injectable solutions are prepared by incorporating the nanoparticles in the required amount in the appropriate solvent with various of the other ingredients enumerated above, as required, followed by sterilization.

2. Routes of Administration

Upon formulation, nanoparticles will be administered in a manner compatible with the dosage formulation and in such amount as is therapeutically effective.

The nanoparticles can be administered to the subject using any method known to those of ordinary skill in the art. For example, a pharmaceutically effective amount of a composition comprising nanoparticles may be administered intravenously, intracerebrally, intracranially, intrathecally, into the substantia nigra or the region of the substantia nigra, intradermally, intraarterially, intraperitoneally, intralesionally, intratracheally, intranasally, topically, intramuscularly, intraperitoneally, subcutaneously, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990). In particular embodiments, the composition is administered to a subject using a drug delivery device.

3. Dosage

A pharmaceutically effective amount of the nanoparticles is determined based on the intended goal, for example inhibition of cell death. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

For example, a dose of the therapeutic agent may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about hours, about 10 hours to about 24 hours, about 1 day to about 2 days, about 1 week to about 2 weeks, or longer, or any time interval derivable within any of these recited ranges.

In certain embodiments, the method may provide a continuous supply of a pharmaceutical composition to the patient. This could be accomplished by catheterization, followed by continuous administration of the therapeutic agent. The administration could be intra-operative or post-operative.

B. Combination Treatments

Certain embodiments of the present disclosure provide for the administration or application of one or more secondary forms of therapies for the treatment or prevention of a disease. For example, the disease may be a hyperproliferative disease, such as cancer.

The secondary form of therapy may be administration of one or more secondary pharmacological agents that can be applied in the treatment or prevention of cancer.

If the secondary therapy is a pharmacological agent, it may be administered prior to, concurrently, or following administration of the nanoparticles.

The interval between the administration of the nanoparticles and the secondary therapy may be any interval as determined by those of ordinary skill in the art. For example, the interval may be minutes to weeks. In embodiments where the agents are separately administered, one would generally ensure that a long period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations the time period for treatment may be extended, however, where several days (2, 3, 4, 5, 6 or 7) to several weeks (1, 2, 3, 4, 5, 6, 7 or 8) lapse between the respective administrations. In some embodiments, the timing of administration of a secondary therapeutic agent is determined based on the response of the subject to the nanoparticles.

Various combinations may be employed. For the example below a CLCN composition is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present disclosure to a patient will follow general protocols for the administration of such compounds, taking into account the toxicity, if any, of the agents. Therefore, in some embodiments there is a step of monitoring toxicity that is attributable to combination therapy. It is expected that the treatment cycles may be repeated. It also is contemplated that various standard therapies, as well as surgical intervention, may be applied in combination with the described therapy.

In specific aspects, it is contemplated that a standard therapy will include chemotherapy, radiotherapy, immunotherapy, surgical therapy or gene therapy and may be employed in combination with the inhibitor of gene expression therapy, anticancer therapy, or both the inhibitor of gene expression therapy and the anti-cancer therapy, as described herein.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa: ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azascrine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU): folic acid analogues, such as denopterin, pteropterin, and trimetrexate: purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine: androgens, such as calusterone, dronanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid: eniluracil; amsacrine: bestrabucil; bisantrene: edatraxate; defofamine; demecolcine: diaziquone; elformithine, elliptinium acetate; an epothilone; etoglucid: gallium nitrate: hydroxyurea: lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid: 2-ethylhydrazide; procarbazine; PSKpolysaccharide complex; razoxane: rhizoxin: sizofiran; spirogermanium: tenuazonic acid; triaziquone: 2,2′,2″-trichlorotriethylamine; trichothecenes (especially T-2 toxin, verracurin A, roridin A and anguidine); urethan; vindesine; dacarbazine; mannomustine: mitobronitol; mitolactol; pipobroman; gacytosine: arabinoside (“Ara-C”); cyclophosphamide: taxoids, e.g., paclitaxel and docetaxel gemcitabine: 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1): topoisomerase inhibitor RFS 2000; difluorometlhylomithine (DMFO); retinoids, such as retinoic acid; capecitabine, carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, may rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment. As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell may bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds; cytokine therapy, e.g., interferons α, β, and γ, IL-1, GM-CSF, and TNF: gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication No. WO2015016718: both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898 and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335, CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752, WO 00/37504 (CP675,206, also known as tremelimumab; formerly ticilimumab), U.S. Pat. No. 6,207,156, can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114, all incorporated herein by reference.

An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752 all incorporated herein by reference, and immunoadhesions such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. Further examples can therefore be contemplated. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. KITS

In various aspects of the embodiments, a kit is envisioned containing therapeutic agents and/or other therapeutic and delivery agents. In some embodiments, the present embodiments contemplates a kit for preparing and/or administering a CLCN composition of the embodiments. The kit may comprise one or more sealed vials containing any of the pharmaceutical compositions of the present embodiments. The kit may include, for example, CLCNs as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### V. EXAMPLES

The following examples are included to demonstrate preferred embodiments of the present disclosure. It should be appreciated by those of skill in the art that the techniques disclosed in the examples which follow represent techniques discovered by the inventor to function well in the practice of the present methods and compositions, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1—Cationic Liquid Crystalline Nanoparticles

Generation and Characterization of CLCNs:

Cationic liquid crystalline nanoparticles (CLCNs) (FIG. 1), were prepared by mixing together a lipophilic phase with a hydrophilic phase with use of high-speed homogenization. The lipophilic phase was made of a cationic phospholipid such as 2-dioleoyl-3-trimethylammonium-propane (chloride salt) (DOTAP) to promote retention of the negatively charged RNAi in the core through electrostatic interaction and to control release of the RNAi and glyceryl monooleate such as 1-(cis-9-octadecenoyl)-rac-glycerol (GMO) to facilitate efficient interaction and fusion with the cell membrane. The hydrophilic phase was prepared dissolving in UltraPure DNase/RNase-Free Distilled Water a nonionic surfactant such as Pluronic F-127 to increase sustained release and to reduce degradation or dissociation of the CLCNs. After the homogenization and the purification, CLCNs were conjugated with nucleic acids, such as siRNA or miRNA therapeutics dissolved in UltraPure DNase/RNase-Free Distilled Water. A 1:1 volume ratio between a calculated concentration of CLCNs and RNAi was used for both in vitro and in vivo experiments.

CLCN1 and CLCN2:

One of the major problem in the use of cationic nanoparticles is the high toxicity in vitro and in vivo due to the high concentration of cationic lipid such as DOTAP used to have positive charged nanoparticles, able to bind with the negative charged RNAi. To moderate DOTAP toxicity (Lv et al., 2006), two CLCN formulations, CLCN1 and CLCN2, were prepared based on the same reagents but with different molar ratios between GMO and DOTAP. Basically, the formulation CLCN1 had a higher DOTAP percentage of ˜18% (wt) than the formulation CLCN2 where the DOTAP percentage was of ˜7% (wt). For both formulations, the Pluronic concentration was 0.5% (w/v). CLCN1 and CLCN2 were tested in the same in vivo and in vitro experiments to check the favorable combination to enable efficient delivery and low toxicity.

Transmission Electron Microscopy Analysis (7) Analysis:

Morphological investigation was performed with a Transmission Electron Microscopy (TEM) operating at 80 kV (FIGS. 2A, B). TEM is a vital characterization tool for directly imaging nanomaterials to obtain quantitative measures of particle and/or grain size, size distribution, and morphology. A few microliters of the CLCN formulations were scanned using magnifications of 200000× and resolution of 100 nm and the images were recorded. FIG. 2A shows the formulation CLCN1 alone and conjugated with the siRNA. FIG. 2B shows the formulation CLCN2 alone and conjugated with the siRNA. CLCN1 and CLCN2 alone and conjugated with siRNA appear monodisperse systems with no sign of agglomeration but in both CLCNs conjugated with the siRNA homogenous and round spheres and core-shell structures are distinguishable. On the basis of these micrographs the following hypothesis may be drawn: the lighter color in the middle of the CLCN1-siRNA and CLCN2-siRNA may indicate the presence of the water channels containing the siRNA.

Physicochemical Characterization of CLCNs:

The quantitative physicochemical characterization of CLCNs was conducted with use of dynamic light scattering (DLS) to determine the size and homogeneity of the CLCNs (FIG. 2C), and a Zetasizer Nano Z to measure the zeta potential (Charge) of the nanoparticles surface (FIG. 2C). The physicochemical analysis revealed that CLCNs alone have a diameter ranging from 60 to 100 nm, with CLCN1 's at about 70 nm and CLCN2's at about 90 nm. CLCNs conjugation with siRNA did not affect overall particle size, but CLCN2 conjugated with siRNA showed larger size (at about 100 nm) than CLCN1 conjugated with same siRNA (at about 90 nm) (FIG. 2C size). CLCNs were homogeneous and stable nanoparticles, as demonstrated by a very low polydispersity index (PDI) ranging from 0.10 to 0.20 (FIG. 2C). In particular, both formulations displayed a lower PDI when conjugated with siRNA, confirming homogenous and monodisperse shape and structure shown in the TEM analysis (FIG. 2A, B PDI). The positive charge on the CLCN surface was between +25 and +35 mV. For CLCN1 and CLCN2 alone, the surface charges were, respectively, ˜+35 and ˜+30 mV (FIG. 2C zeta potential). When CLCN1 and CLCN2 were conjugated with siRNA, the surface charges were, respectively, ˜+30 and ˜+45 mV (FIG. 2C zeta potential). If all of the particles in suspension have a large positive zeta potential, they will not tend to aggregate or to flocculate. Particles with zeta potentials that are more positive than +30 mV are normally considered stable. The surface charge did not change from positive to negative after siRNA conjugation, suggesting complete internalization of the RNAi within the hydrophilic core and a stable nanoparticle suspension (FIG. 2C zeta potential). The amount of RNAi conjugated to the CLCNs was measured using a red fluorescent siRNA (Cy5). Briefly, after the conjugation process the CLCNs-siRNA Cy5 were placed in 3K ultra centrifugal filter unit and centrifuged. The ultra-filtrate contained the free siRNA Cy5 was measured at a wavelength of excitation 650 nm and emission 670 nm. A standard curve was used to determinate the amount of siRNA form the fluorescence intensity. The amount of siRNA Cy5 conjugated to the CLCNs was calculated subtracting the amount of siRNA Cy5 added during the preparation procedure to the amount of free siRNA Cy5 found after the centrifugation. The results was around 80% for both formulations. (FIG. 2C amount siRNA Cy5 conjugated (%)).

Evaluation of the Retardation of RNAi by CLCNs:

Gel retardation assays were performed to evaluate the nanoparticle retardation inside the gel and the siRNA condensation inside the CLCNs (FIG. 2D). Electrophoresis in 1% Agarose gels were carried out at 100 V for 20 minutes. A calculated amount of free siRNA was used as standard control and the same concentration was conjugated to the CLCN formulations (CLCN1-siRNA and CLCN2-siRNA); CLCN formulations alone were also loaded in the gel (CLCN1 and CLCN2). After electrophoresis, the gels were analyzed with use of a gel imaging system. The relative density of the bands was calculated to quantify the nanoparticles retardation inside the gel and the siRNA condensation inside the CLCNs, using ImageJ software (1.46r). Basically, the percentage of the Area for each peaks resulted from the Agarose gel analysis was calculated and the Percent value for each sample (CLCN1 and CLCN2; CLCN1-siRNA and CLCN2-siRNA) was divided by the Percent value for the standard (free siRNA) to obtain the relative band density (fold change value). The condensation of siRNA inside the CLCNs was around 80% for both formulations indicating that the binding between the carrier and the siRNA was strong enough to withstand dissociation during electrophoresis, whereas the siRNA not complexed into CLCNs was free to run on the bottom of the agarose gel (FIG. 2D).

Nanoparticle Tracking Analysis (NTA):

To visualize, measure, and count the nanoparticles a Nanoparticle tracking analysis (NTA) was performed (FIG. 2E, F) (Table 1). In this analysis, each nanoparticle in solution is individually but simultaneously analyzed by direct observation and measurement of diffusion events, producing high-resolution results for particle size distribution and concentration (Filipe et al., 2010). The mean size values obtained with the NTA were in the same range as those obtained by DLS analysis. NTA showed a higher particle concentration when CLCNs were alone. Specifically, CLCN1's concentration was ˜7.23e+008 particles/ml, and CLCN2's was ˜5.05e+008 particles/ml (FIG. 2E, F) (Table 1); however, CLCNs complexed with siRNA displayed a lower concentration: CLCN1-siRNA's was 3.82e+008 particles/ml, and CLCN2-siRNA was 1.69e+008 particles/ml (FIG. 2E, F) (Table 1).

TABLE 1
Mean size and size distribution of CLCNs from NTA. Mean and Standard
Deviation (SD) calculated by NTA software; Concentration (CONC.)
in particles E8/ml as measured by NTA. Numbers represent average
values ± standard deviation (n = 3 measurements).

| | | | PARTICLE |
|---|---|---|---|
| FORMULATION | MEAN(nm) | SD (nm) | CONC. (E8/ml) |
| CLCN1 | 64 9 ± 1.1 | 27.0 ± 2.3 | 7.23 |
| CLCN2 | 99.1 ± 3.5 | 36.3 ± 2.6 | 5.05 |
| CLCN1-siRNA | 95.2 ± 6.8 | 49.8 ± 10.4 | 3.82 |
| CLCN2-siRNA | 129.9 ± 4.8 | 40.1 ± 4.1 | 1.69 |

Cellular Uptake and Processing of CLCNs in H1299:

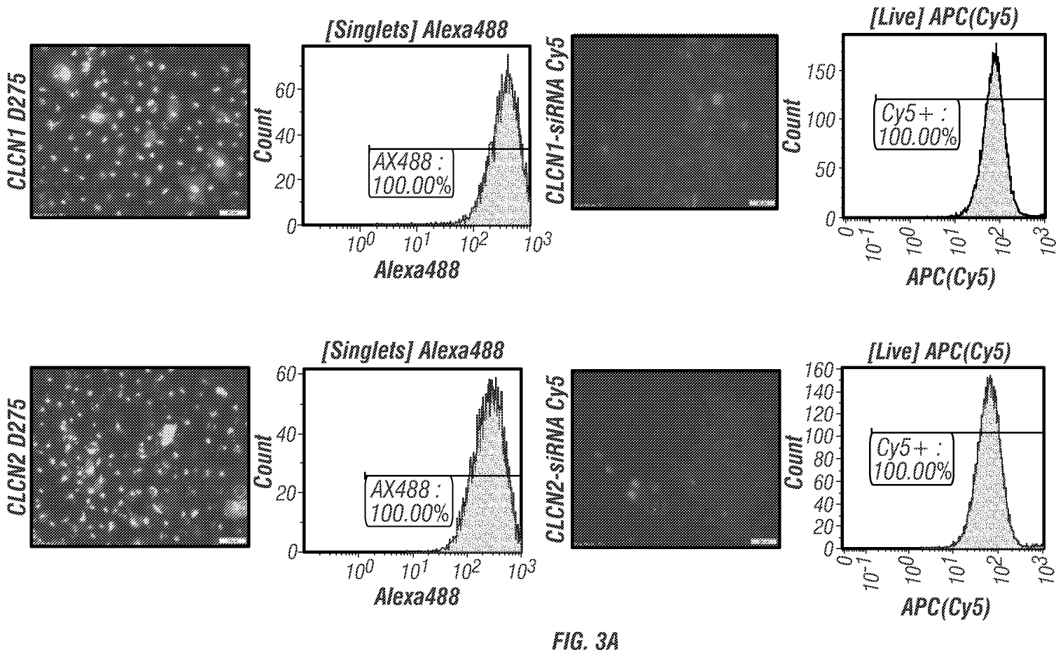

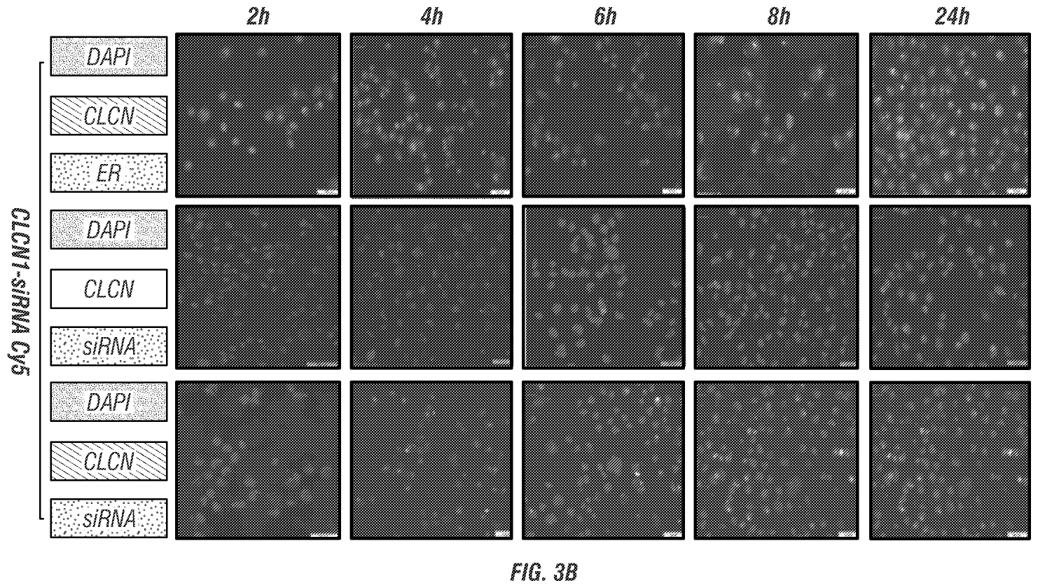

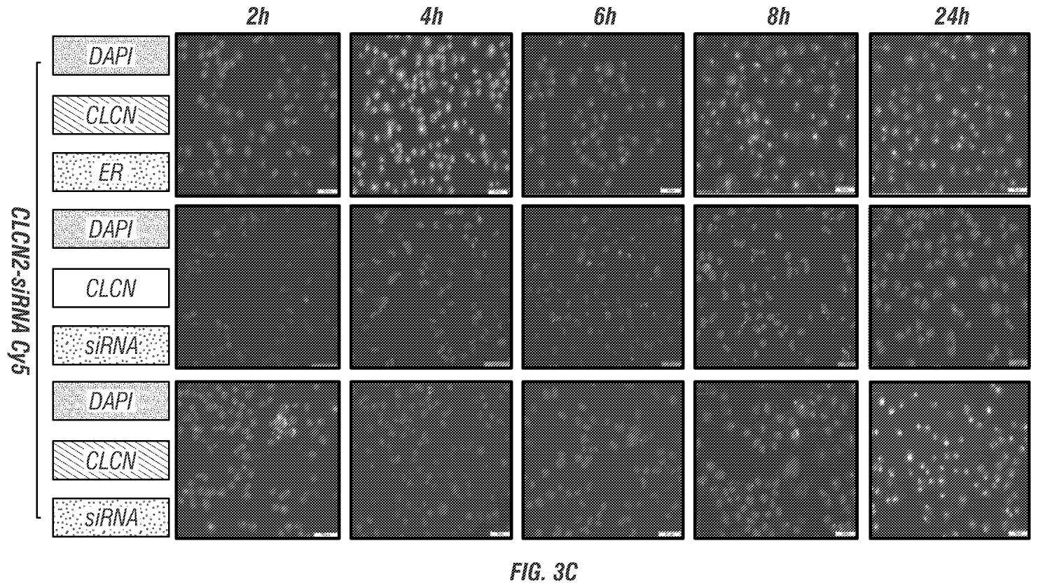

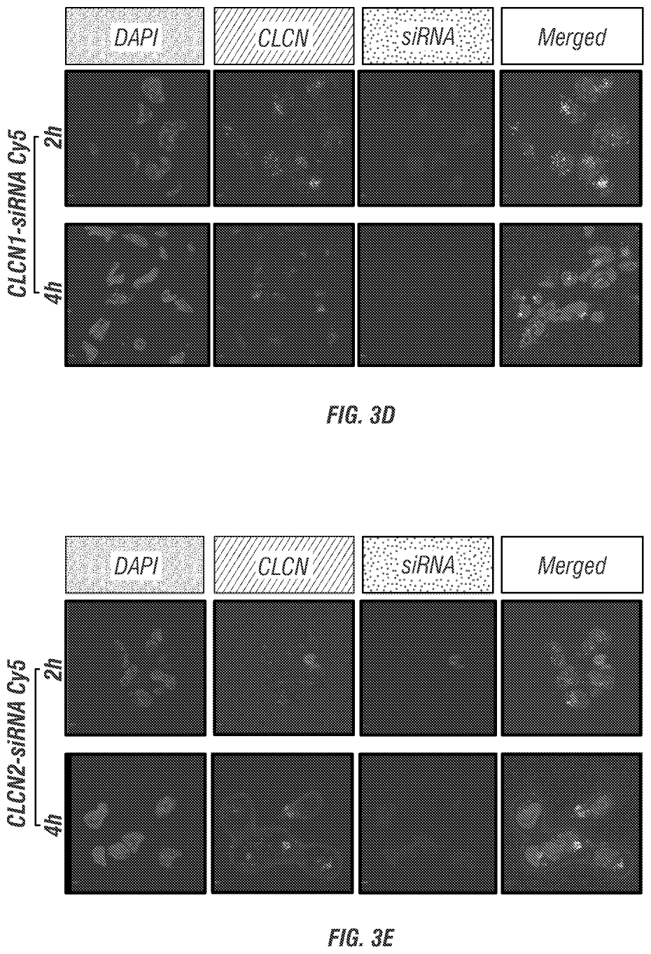

The kinetics of internalization and intracellular trafficking of nanoparticle formulations were subsequently analyzed. As readout for monitoring the delivery of CLCNs, D275 fluorescent nanoparticles were prepared by using a fluorescent lipophilic tracer in the lipophilic phase and fluorescent and not fluorescent CLCNs were complexed with Cy5 fluorescent siRNA. In FIG. 3A the quantification of the Fluorescent signal intensity was quantified by flow cytometry analysis and fluorescence microscopy images were taken, 24 hours after treatment on H1299 cell lines, the green fluorescent CLCNs 1 and 2 were able to diffuse into cells and release red fluorescent siRNA in the cytoplasm (FIG. 3A).

Fluorescence Microscopy Analysis:

To better analyze the kinetics of cellular uptake and processing of the CLCNs, fluorescence microscopy images were taken at 2, 4, 6, 8, and 24 hours after treatment (FIGS. 3B, C). Green fluorescent (D275) CLCNs were prepared and fluorescent and not fluorescent CLCNs were complexed with red fluorescent (Cy5) siRNA. Various markers were used to determine the localization of CLCNs for each time point (FIG. 3B, C). In the first line in FIGS. 3B and C, H1299 cells were treated with green fluorescent CLCNs D275 and at each time point after treatment stained for the nucleus (DAPI) and endoplasmic reticulum (ER, ER-Tracker™). Both formulation were able to diffuse in the cytoplasm but the formulation CLCN2 (FIG. 3C) escaped faster from the ER than the formulation CLCN1 did (FIG. 3B). In the second line in FIGS. 3B and 3C, to analyze the kinetics of siRNA release in the cytoplasm, cells were treated with no fluorescent CLCNs complexed with fluorescent siRNA-Cy5 and counterstained for the nucleus with DAPI. The fluorescent signal of siRNA-Cy5 was already visible after 2 hours and increased over time in both formulations (FIGS. 3B, C). Finally in the third line, cells were treated with D275 fluorescent CLCNs complexed with Cy5 fluorescent siRNA and counterstained for the nucleus with DAPI. Data showed D275 fluorescent CLCNs and the Cy5 fluorescent siRNA were dispersed in the cytoplasm after 2-4 hours (FIG. 3B, C) as the previous fluorescence microscope images displayed.

Confocal Microscopy Images:

Confocal microcopy images were performed to confirm the mechanism of uptake and internalization of the CLCNs and the release of siRNA in the cytoplasm. CLCNs were labeled with D275 fluorescent lipophilic tracers and conjugated with Cy5 fluorescent siRNA. H1299 tumors cells were treated for 2 and 4 hours with CLCNs D275-siRNA Cy5. After 2 and 4 hours DAPI was used to label the nucleus and confocal images were taken. In FIGS. 3D and 3E single color channels and all channels overlapped together in one single image (merged) are shown. The confocal images confirmed the results previously reported at the same time points with the fluorescence microscopy, the CLCNsD275 were dispersed in the cytoplasm and able to release the siRNA Cy5 2 hours after treatment on H1299 cells (FIGS. 3D, E). These results suggest that CLCNs are able to deliver siRNA after interaction with the cellular membrane and release it in the cytoplasm as early as 2 hours after treatment.

Intracellular Trafficking of CLCNs in H1299 Cells by TEM:

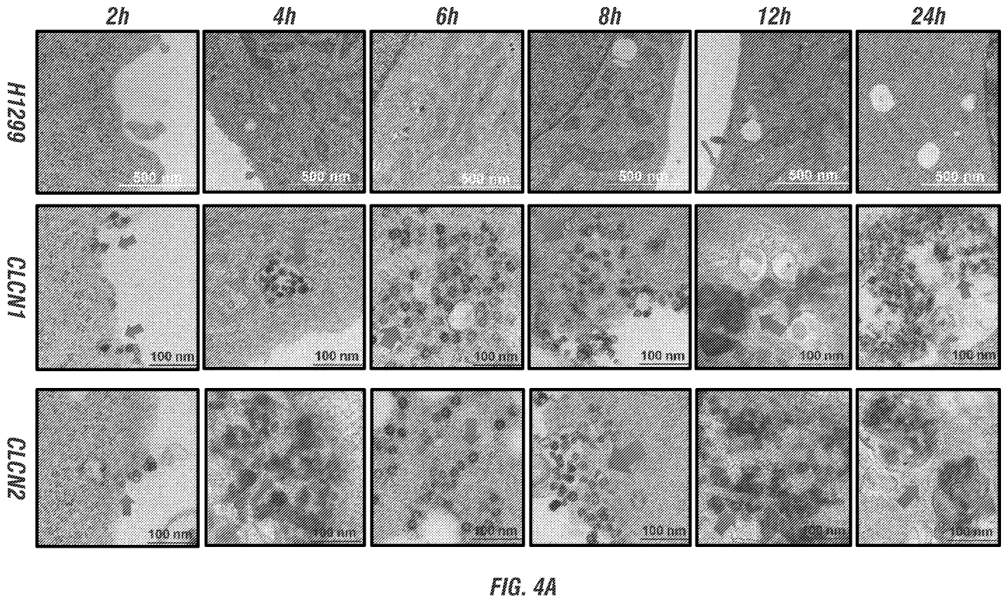

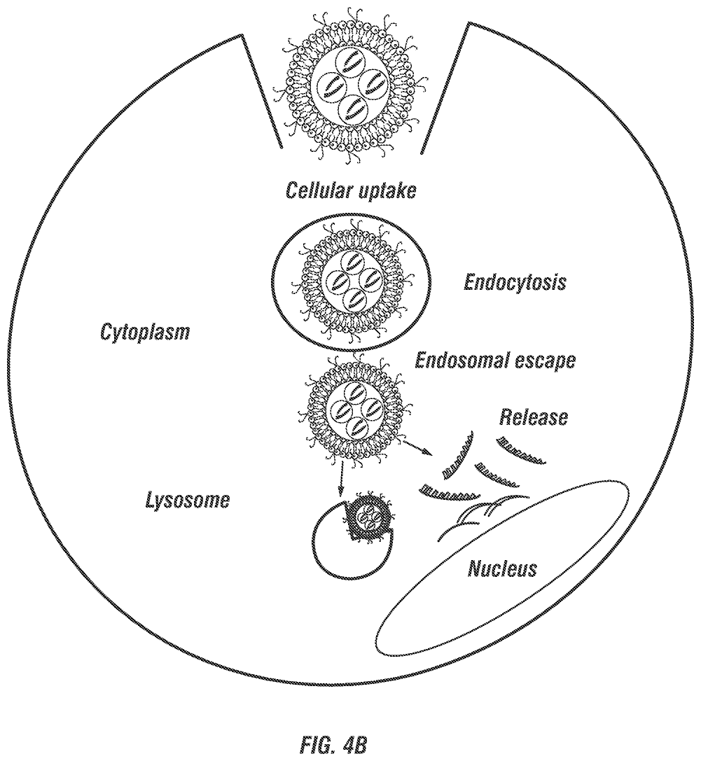

Further intracellular trafficking analysis conducted by TEM (FIG. 4A) showed that CLCNs are taken up through endocytosis upon binding with the cell membrane and travel from early endosomes to the lysosome after 24 hours. The results also highlighted that CLCNs adhering to the plasma membrane were subsequently internalized by a vesicle-mediated endocytosis process. Nanoparticles located outside the endosomes were also observed at 6 hours and 8 hours. This further emphasizes the ability of CLCNs to escape endolysosomal entrapment shortly after intracellular uptake (FIG. 4A). Thus, CLCNs are able to get inside the cells, escape from the ER, and release siRNA in the cytoplasm (FIG. 4B).

CLCNs RNAi Mediated Gene-Silencing and Gene-Expression Evaluation In Vitro:

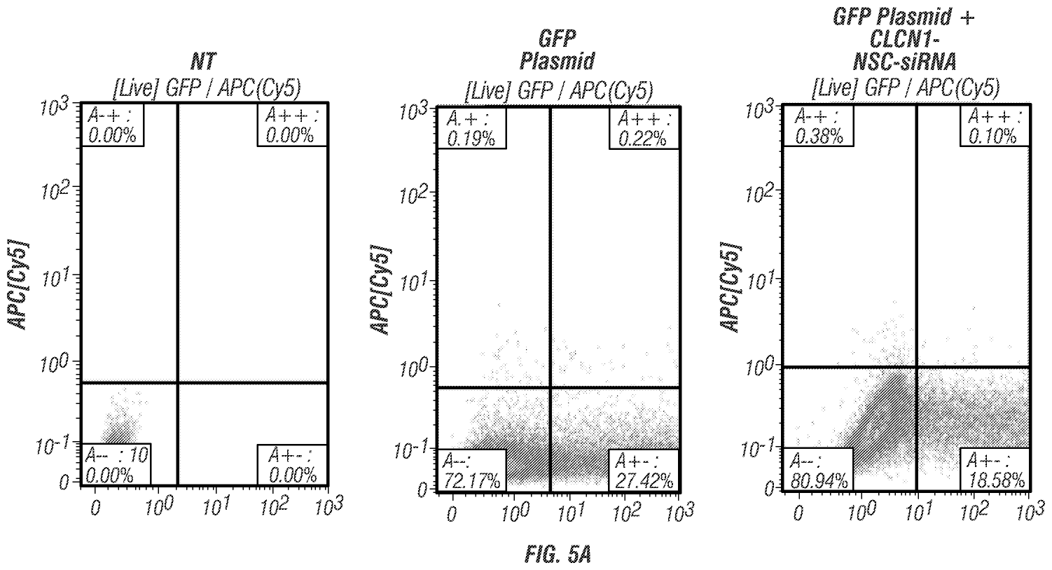

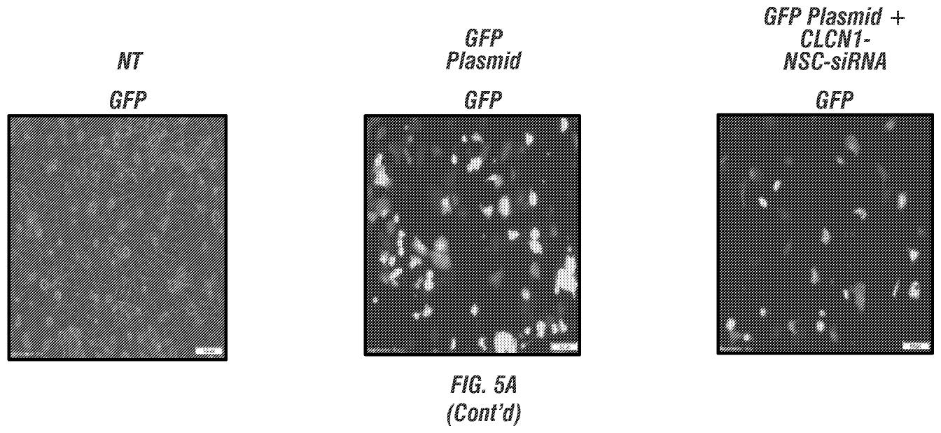

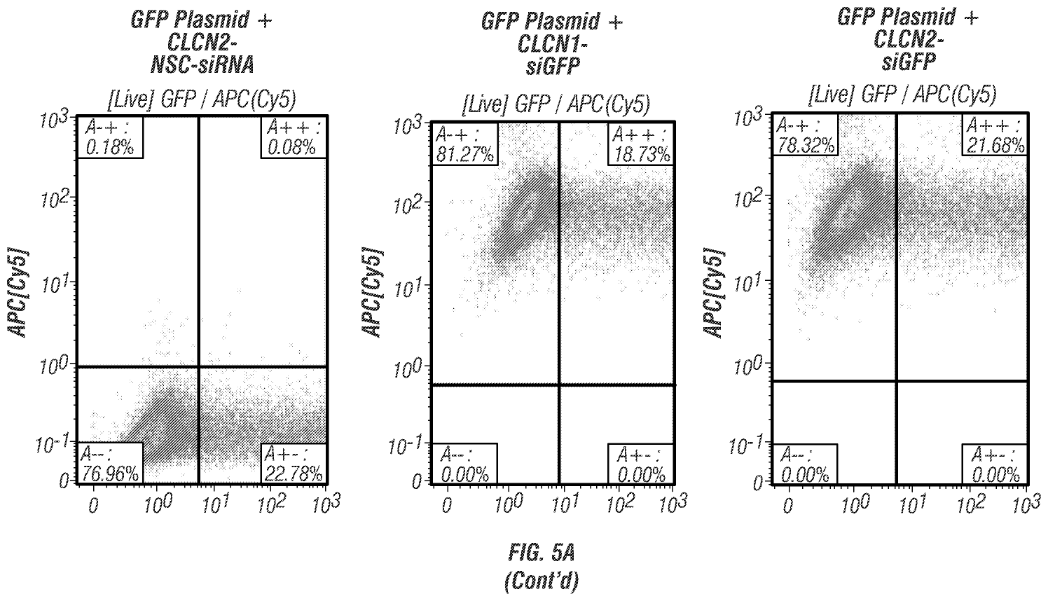

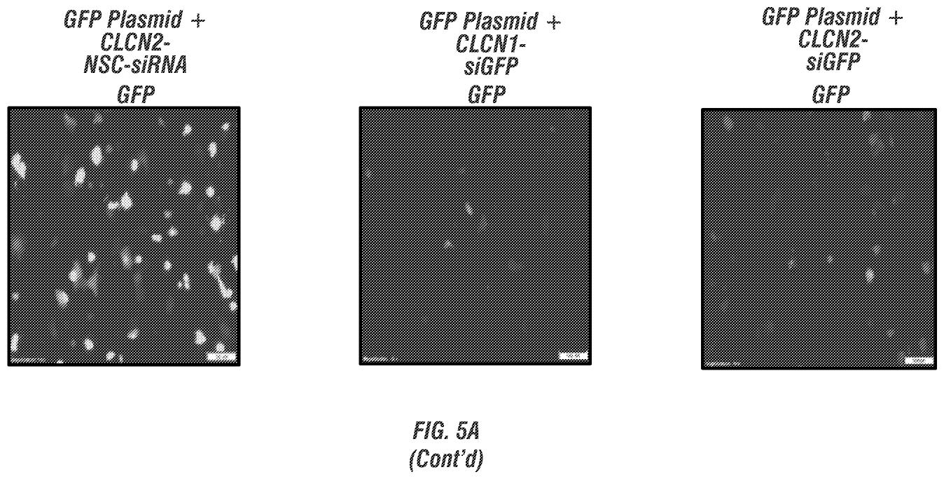

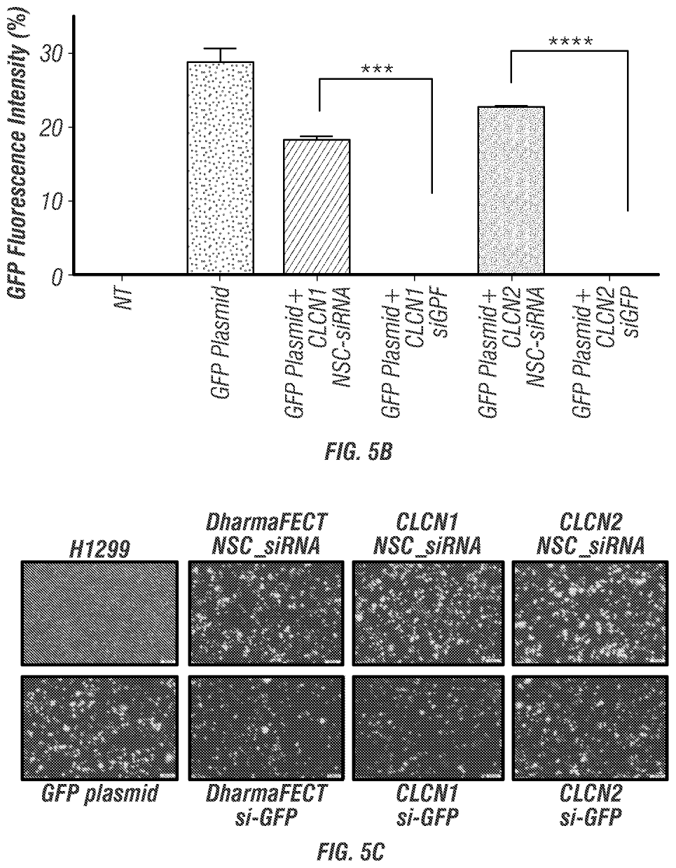

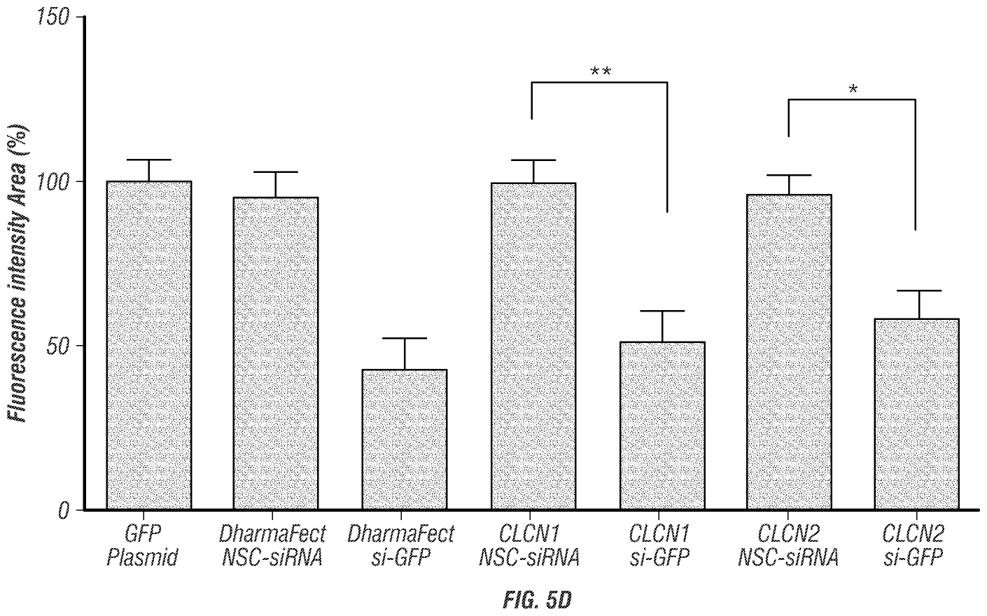

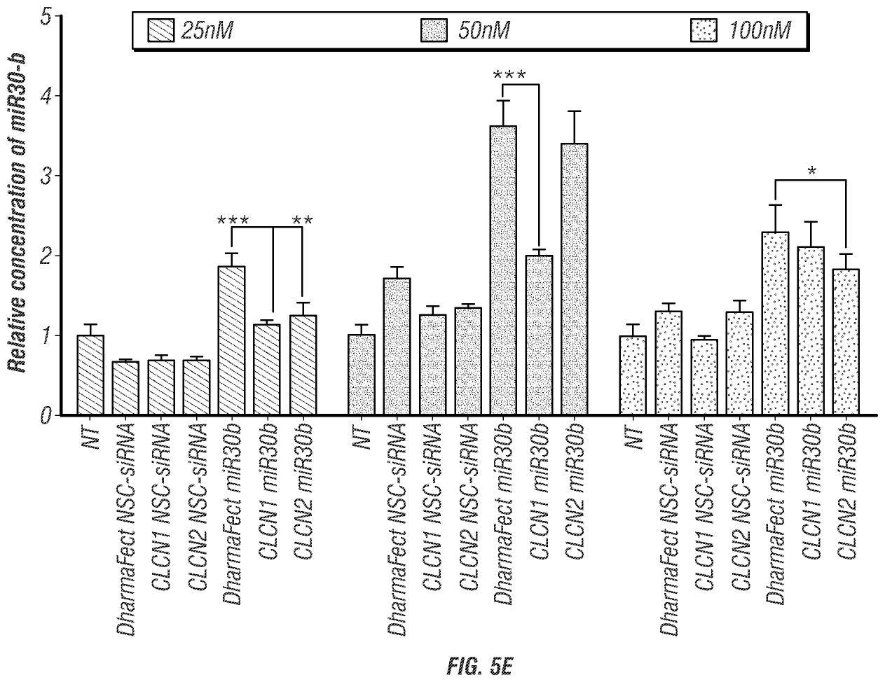

Gene-silencing and gene expression evaluation experiments were performed to determine whether CLCNs are able to deliver siRNA to cells to induce silencing of a reporter gene (Green fluorescent protein. GFP) or enhancing the expression of an endogenous microRNA (miR-30b) (FIG. 5). To this purpose, in the gene-silencing experiments, H1299 cells were cotransfected for 24 hours with a GFP plasmid conjugated with Lipofectamine 2000 and CLCNs/anti-GFP siRNA (si-GFP) complexes or CLCNs/NSC-siRNA as negative control siRNA, and flow cytometry and fluorescence microscopy analyses were conducted. When the H1299 were transfected with the GFP plasmid only or cotransfected with the GFP Plasmid and the CLCNs/NSC-siRNA, a high GFP transfection efficiency was achieved, whereas in the cells cotransfected with the GPF plasmid and CLCNs/anti-GFP siRNA (si-GFP) complexes, the GFP fluorescence intensity measured by flow cytometry analysis was dramatically reduced (***) p value 0.0005 and (****) p value <0.0001 (FIG. 5A, B). The experiment was repeated two times and the silencing efficiency of CLCNs was compared by fluorescence microscopy with that of the commercial transfection reagent DharmaFect (Dharmacon) (FIG. 5C. D). Both CLCN formulations were able to deliver the anti-GFP siRNA and inhibit the GFP transfection as the DharmaFect did.

Gene expression evaluation in vitro was conducted on H1299 cells transfected with CLCNs conjugated with miR30b and the transfection efficiency was compared with that of the commercial transfection regent DharmaFect (Dharmacon) binding miR30b as well. Basically, H1299 were treated for 24 hours with various concentrations of miR30b, 25. 50 and 100 nM, conjugated with CLCNs or DharmaFect. As a negative control a scramble siRNA was used at the same concentrations and conjugated with CLCNs or DharmaFect. After 24 hours the cells were collected and the miR30b expression was evaluated by qRT-PCR assay. The results showed that CLCNS were able to transfect the cells with miR30b as well as DharmaFect did and the miR30-b expression in vitro was increased by using CLCNs or DharmaFect. Statistical analysis was performed to compare the transfection efficiency of CLCNs-miR30b vs DharmaFect-miR30b. Significantly different p-values were found at 25 nM concentration were the DharmaFect worked better than CLCNs above all the formulation CLCN1-miR30b vs DharmaFect-miR30b=0.0003 (***) showed a lower transfection efficiency results. However, CLCNs showed equivalent transfection efficiency with DharmaFect at higher concentration like 50 and 100 nM. All of these results suggested that CLCNs are able to efficiently transfect the cells in vitro and increase the miR30b expression similar to that seen with DharmaFect.

Biodistribution of CLCNs by Systemic Administration and Effect on Gene Expression in NSCLC Tumor-Bearing Mice:

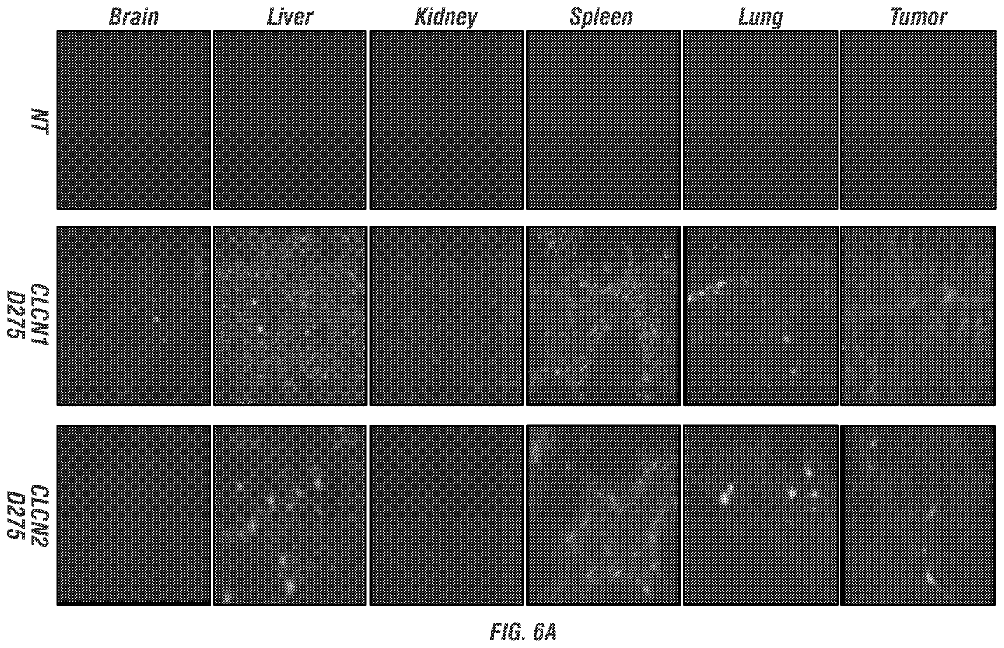

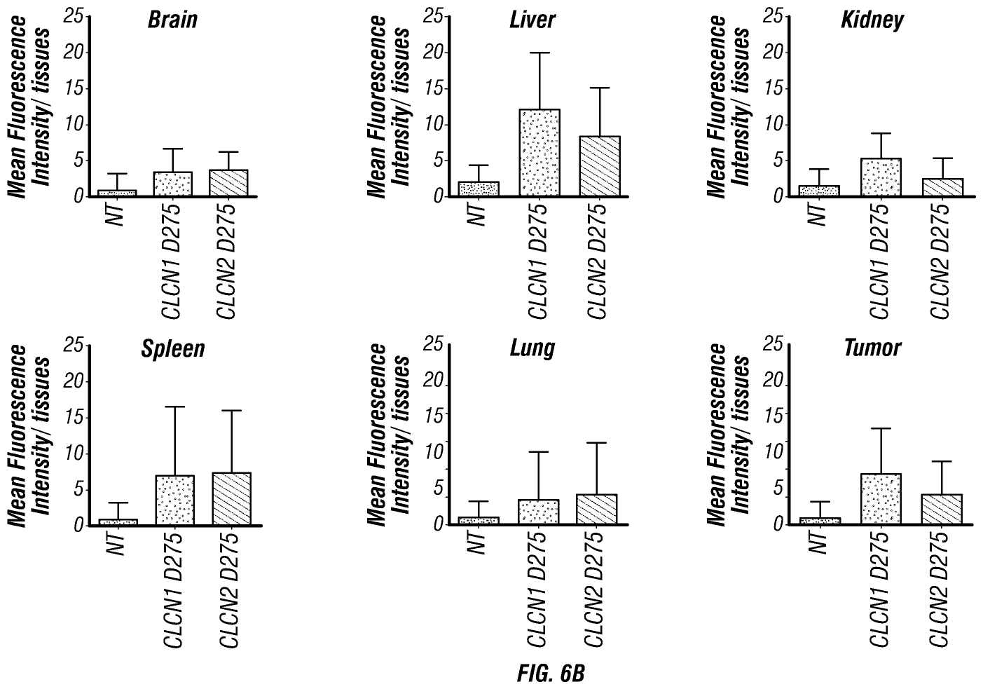

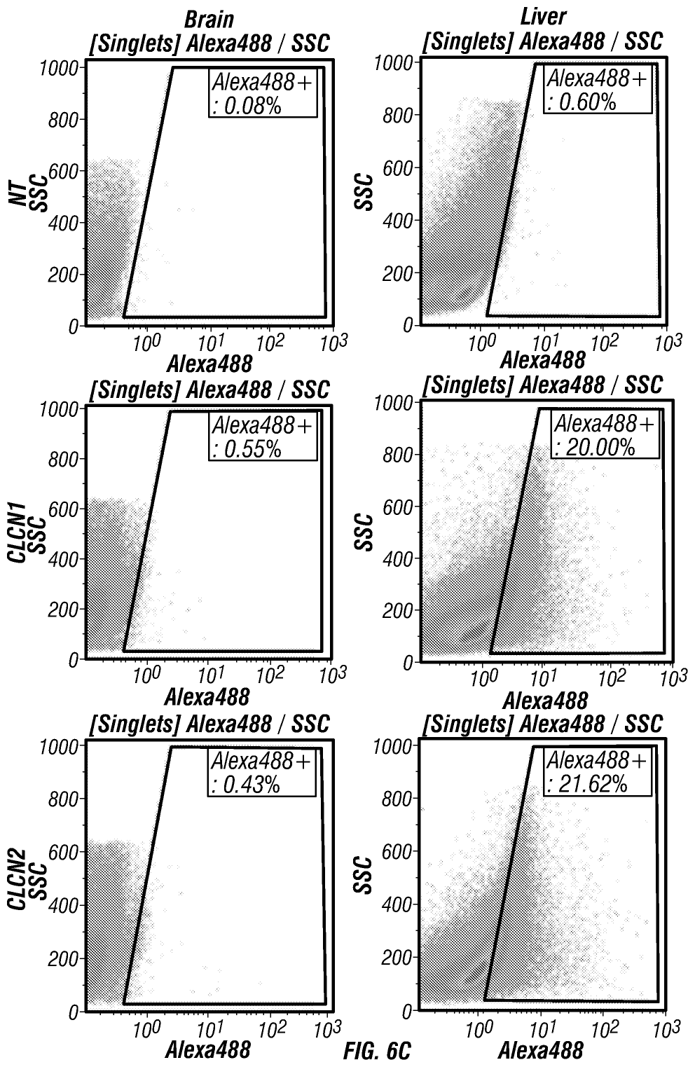

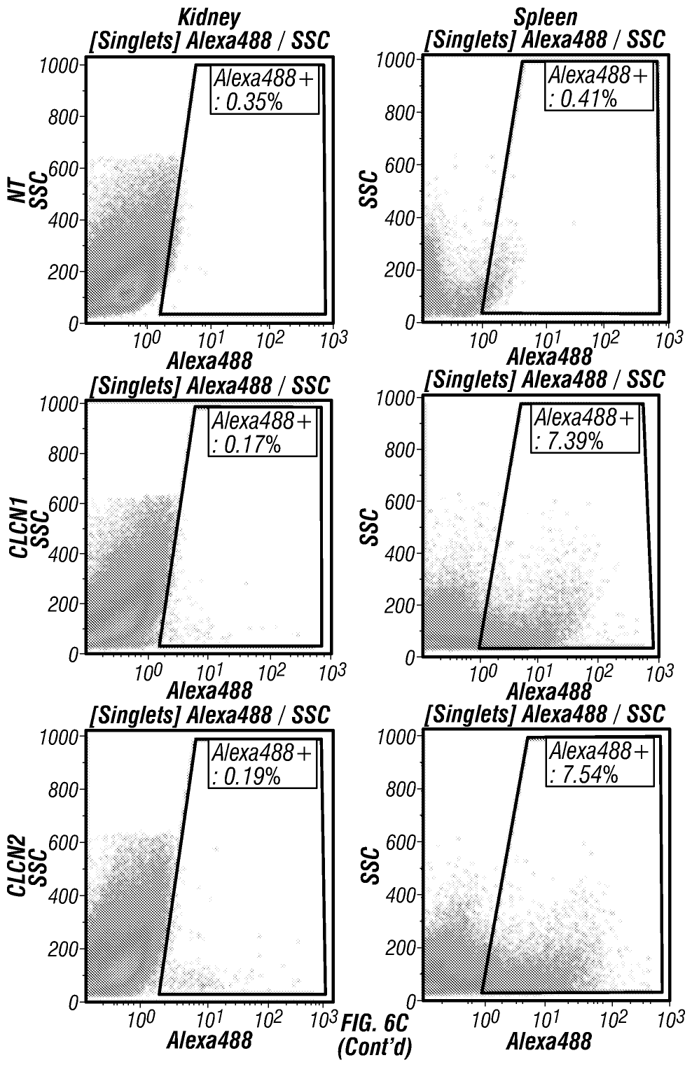

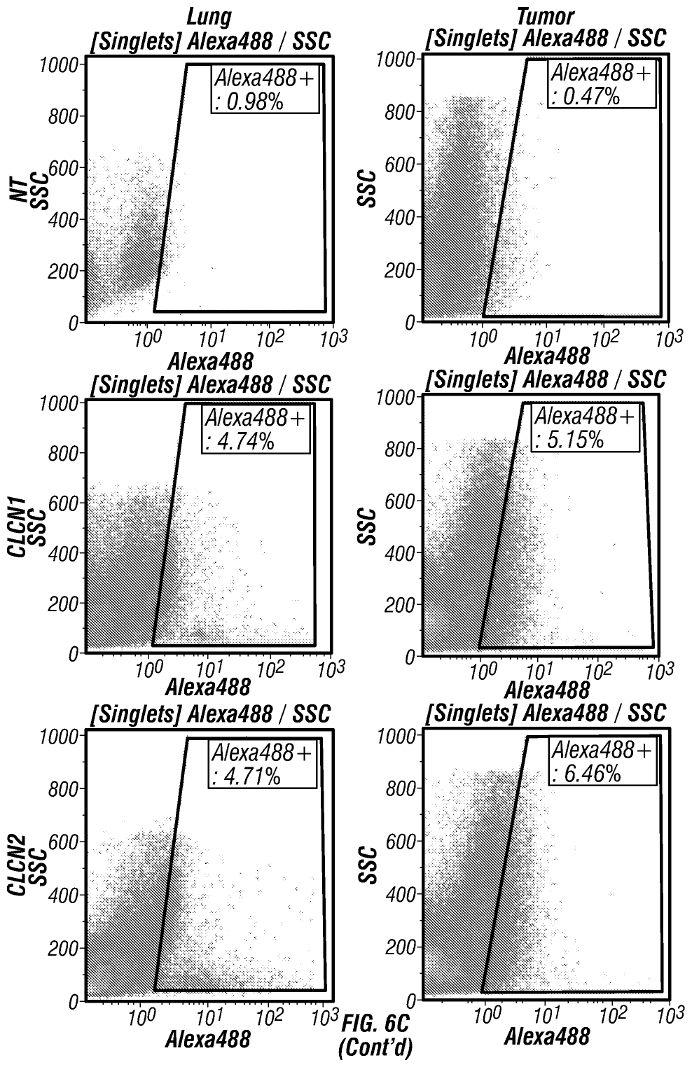

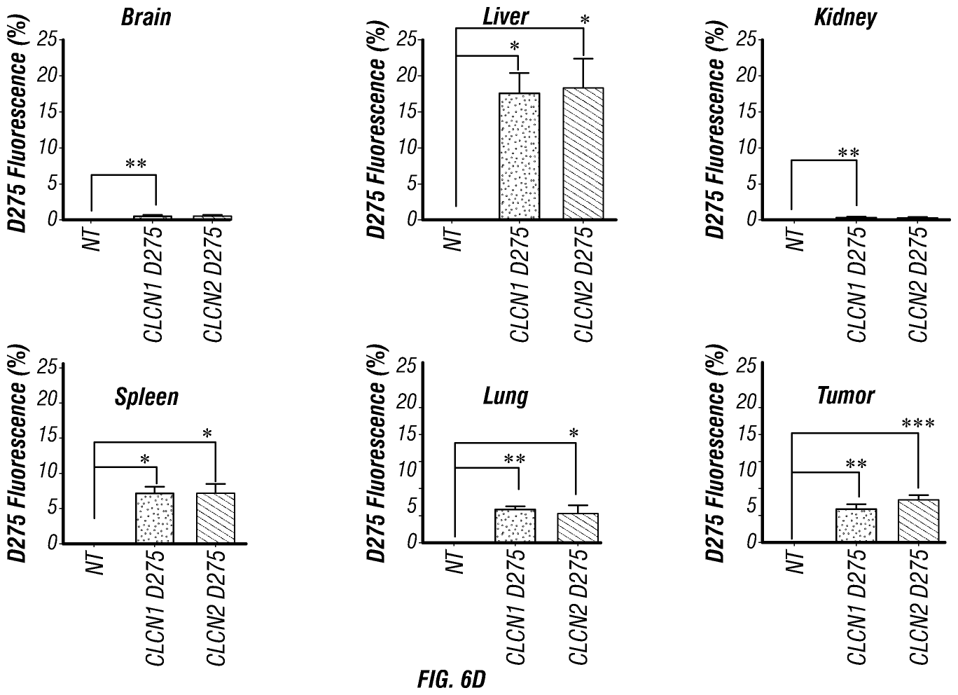

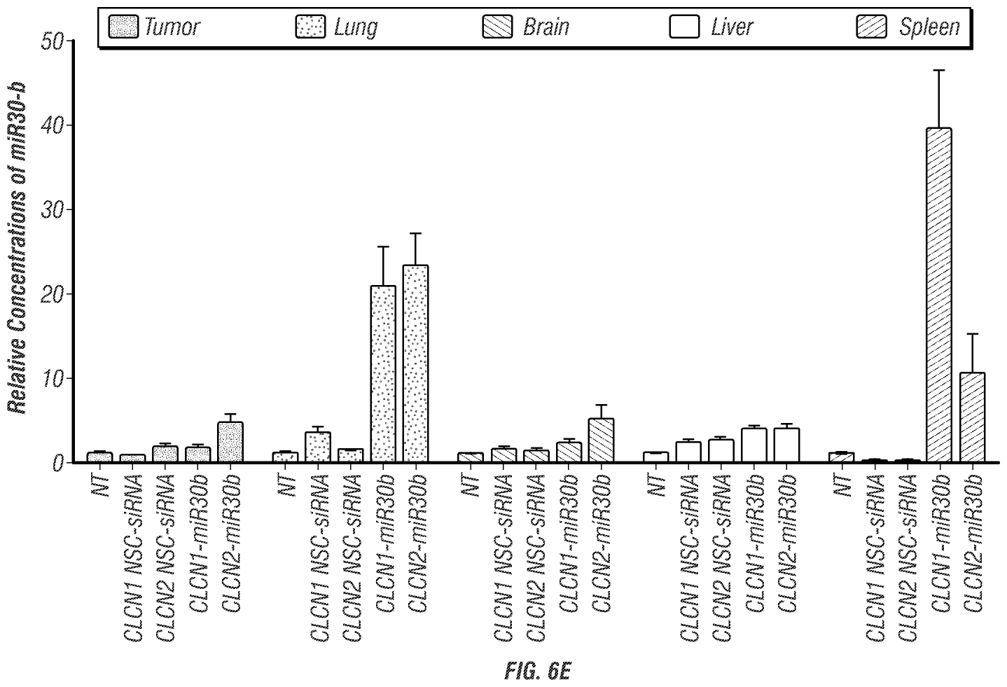

In vive experiments were performed to evaluate CLCNs biodistribution and RNAi delivery. To study CLCNs biodistribution, nu-nu mice with H1299 subcutaneous tumors were injected intravenously with fluorescent CLCN1 D275 and CLCN2 D275 (10 mg/kg), and after 24 hours, the fluorescence intensity of the CLCNs was evaluated in tumors and major organs including liver, spleen, brain, lung, and kidney by fluorescence microscopy and flow cytometry analysis (FIG. 6). Both fluorescence microscopy (FIGS. 6A, B) and flow cytometry analysis (FIGS. 6C, D) showed a higher signal in the liver, spleen, tumor, and lung for both CLCN formulations. In another experiment, the same mice model was used to evaluate the effect on gene expression. CLCNs-miR30b complexes and CLCNs/negative siRNA control complexes were injected at a dose of 1.5 mg/kg via tail vein. Total RNA was extracted from tumors and major organs 24 hours later. The Quantitative real-time PCR showed a high concentration of miR30b in spleen and lung, liver and tumor (FIG. 6E). These results suggest that CLCNs are able to reach the major organs like lung, liver spleen and the subcutaneous tumor and also if conjugated with a microRNA to deliver it to the major organs and increase the expression.

Analysis of Tumor Growth Rate after CLCN2-mR150 Inhibitor Administration:

In this in vivo experiment, a miR150 inhibitor was delivered intravenously using CLCN2 to treat H1299 human lung cancer xenografts. Tumor size was monitored for 3 weeks and statistical analysis of the tumor growth rate was performed using generalized linear mixed models. The tumor growth rate of the CLCN2-miR150 group was lower than that of the control group (1.9% vs 18.0%, p<0.05, Table 2). These studies indicate that CLCN2 were able to efficiently deliver miR150 inhibitor and mediate suppression of tumor growth.

TABLE 2
Estimate of tumor growth rates by treatment groups. Statistical
analysis between the control group and the group treated with
CLCN2-miR150. (n = 5 mice for each group). P < 0.05

| | Slope estimate | Estimated tumor growth rate |
|---|---|---|
| | On log2(tumor size) | On raw scale |
| CLCN2-miRI50 | 0.02667 | 1.9% |
| Control | 0.2393 | 18.0% |

CLCNs Toxicity In Vitro and Evaluation of Damages in Organs Function after CLCNs In Vivo Treatment:

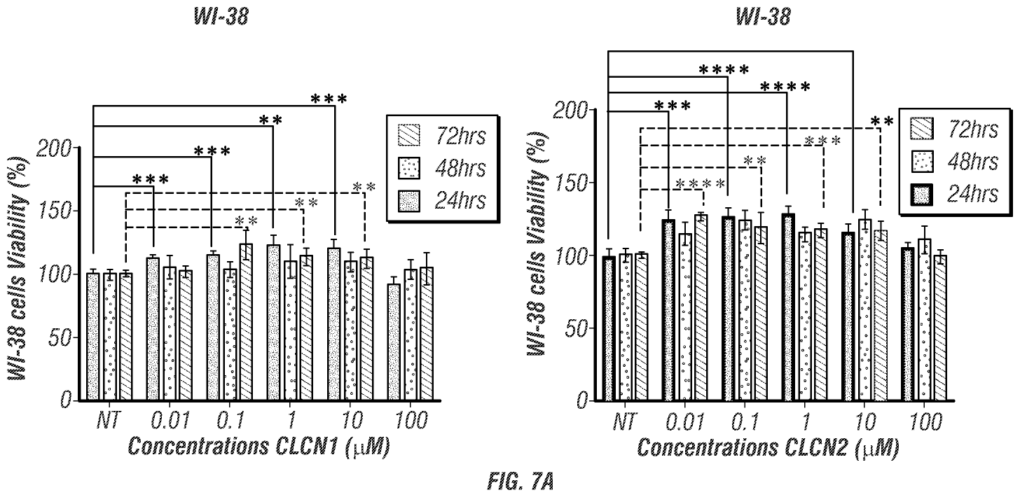

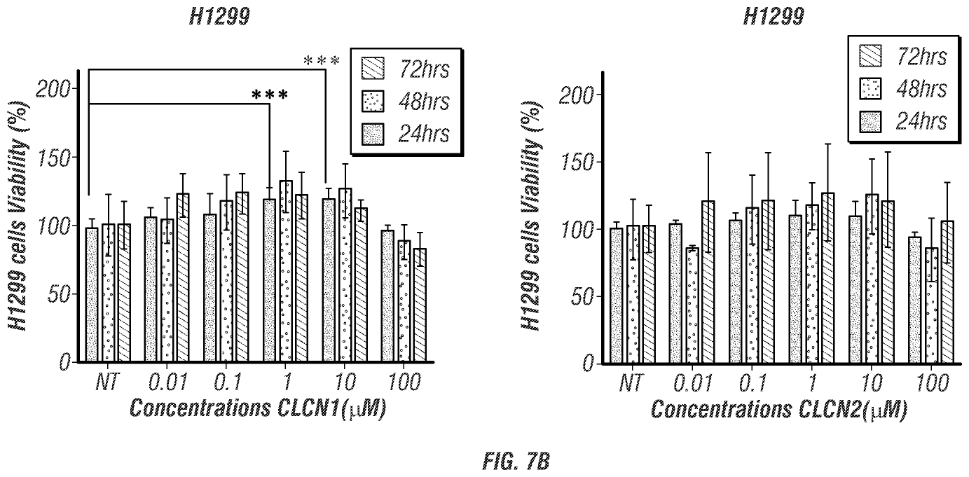

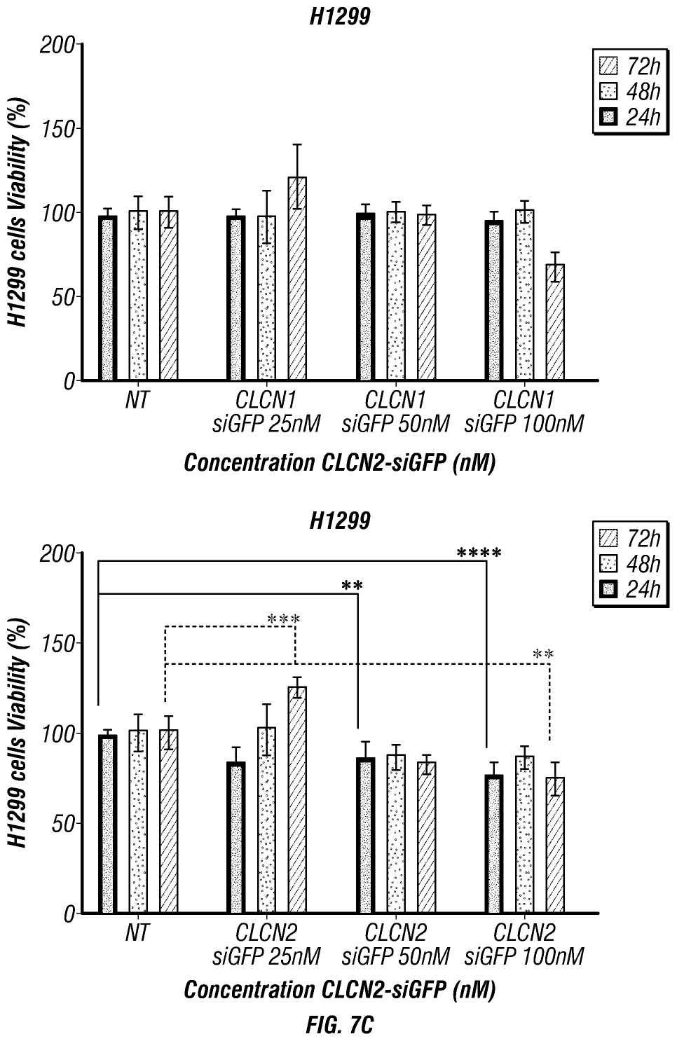

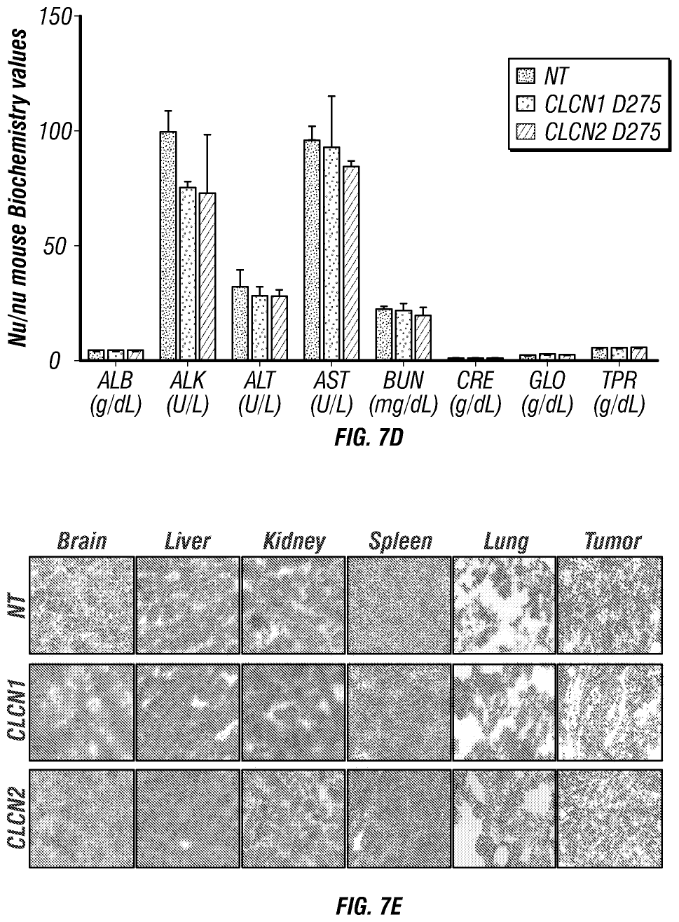

Cytotoxic effects of the nanoparticles were first tested in vitro in lung cancer (H1299) and normal fibroblast and bronchial epithelial cells (WI-38) (FIG. 7). Varying concentrations of CLCNs, from 0.01 to 100 μM, were used to treat the cells, and cell viability and proliferation were evaluated after 24, 48, and 72 hours. The CLCNs were not toxic on normal cells WI-38 (FIG. 7A) or H1299 tumor cells (FIG. 7B). The cytotoxicity of CLCN-siRNA complexes was also evaluated on H1299 tumor cells (FIG. 7C) at varying nM concentrations of siRNA (25, 50, and 100 nM). For in vivo studies, mice were treated with fluorescent CLCN1 D275 and CLCN2 D275 at a dose of 10 mg/kg by intravenous injection. After 24 hours, blood was collected from each mouse for a routine chemistry analysis to check liver or kidney function (FIG. 7D), this analysis showed no liver or kidney damage, thus suggesting that CLCNs are not associated with any changes in hematological parameters or serum biochemical markers. A routine histopathology analysis (FIG. 7E) was performed to check alterations in the major tissues after CLCNs treatment. Specifically, after 24 hours of CLCN1 and CLCN2 injection at 10 mg/Kg dose, all major organs and tissues were collected, and sections were stained for Hematoxylin and eosin stain. A no treatment group was used to compare the tissue anatomy and morphology with the treated groups. Pathologic review showed that there were no abnormalities or changes for all major organs and tissues after exposure of animals to 10 mg/kg dose of CLCN1 and CLCN2. The results obtained from the blood chemistry and the histopathology analysis indicated that the CLCNS are not associated with organ toxicity after 24 hours of treatment at the dose of 10 mg/Kg. Among the two different formulations investigated in this study, CLCN2, having a lower percentage of DOTAP, exhibited better results in term of RNAi delivery and low toxicity in vitro and in vivo.

Thus, the present studies provide CLCNs as monodispersed delivery systems that are about 100 nm in diameter, with a lipid bilayer enclosing an aqueous core, surrounded by a more hydrophobic shell. CLCNs have a positively charged surface, and are able to bind with nucleic acids, such as siRNA or miRNA therapeutics and keep it inside the structure. CLCNs are very safe and biocompatible, even when they were conjugated with RNAi. The tight conjugation may be due to the hydrophobic cationic material and the hydrophobic portion of the amphiphilic material providing a non-polar polymer matrix for loading, protecting, and promoting RNAi molecules retention and controlling the release.

### Example 2—Materials and Methods

Materials:

1-(cis-9-octadecenoyl)-rac-glycerol (monoolein, glyceryl monooleate, GMO content >99%), Pluronic F-127 was purchased from Sigma-Aldrich. 1,2-diolcoyl-3-trimethylammonium-propane (chloride salt) (DOTAP) was purchased from Avanti Polar Lipids. These chemicals were used as received without further purification.

Cells from the H1299 (human non-small cell lung cancer) cell line were cultured in RPMI-1640 medium (HyClone) supplemented with 10% fetal bovine serum (FBS) (HyClone). The Wi-38 (normal bronchial epithelial cells) were cultured in Eagle's Minimum Essential Medium (EMEM) (Corning) supplemented with 10% FBS (Hyclone). Cells were grown at 37° C. in a humidified atmosphere of 5% CO₂ (v/v) in air. Cells were seeded at an initial density of 20%-25% confluence in 6-well plates or 60-mm or 100-mm culture dishes or chamber slides according to experimental procedures and grown for at least 24 hours before any treatment.

Preparation of CLCNs:

1-(cis-9-octadecenoyl)-rac-glycerol and 2-dioleoyl-3-trimethylammonium-propane (chloride salt) were solubilized in ethanol at different molar ratios. 25 mg of Pluronic F-127 was solubilized at 4° C. in RNAsi-free water. The polymeric solution was added drop by drop to the lipophilic mixture under high-speed homogenization (IKA ULTRA-TURRAX T-25). The resulting solution was placed on a magnetic stirring plate for 24 hours for ethanol evaporation, after which the dispersion was stored at 4° C. before further experimentation to enable equilibration of lipids, Pluronic, and water. Green fluorescent CLCNs were prepared by using a lipophilic tracer D275 (Invitrogen molecular probe) in the lipophilic phase at 0.01% (w/v). CLCNs at various molar ratios were conjugated with miRNA (Ambion, ThermoFisher scientific) or siRNA (Sigma-Aldrich) in sterile conditions. Briefly, for both in vitro and in vivo experiments, a 1:1 volume ratio between a calculated concentration of CLCNs and RNAi was used. Red fluorescent siRNA-Cy5 (siRNA Fluorescent Universal Negative Control #1. Cyanine 5 Sigma-Aldrich) was conjugated to CLCNs for imaging experiments.

CLCNs Physicochemical Characterization.

CLCNs and the CLCNs complexed with siRNA or miRNA were analyzed by DLS measurements (ZetaSizer Nano ZS, Malvern Instruments) to retrieve information on size and polydispersion index (PDI), at a temperature of 25° C.±0.1° C. About 20 μl of each nanoparticle suspension was diluted in water, housed in disposable polystyrene cuvettes of 1-cm optical path length, and backscattered by a 4 mW He—Ne laser (operating at a wavelength of 633 nm) at an angle of 173° (each sample was measured 5 to 10 times). The zeta potential was measured by using standard disposable Z potential flow cells after the particles were diluted in water (as neutral charged solution). All measurements were repeated three times at 25° C. The amount of RNAi conjugated to the CLCNs was measured after centrifugation in Amicon Ultra centrifugal Filters 3K (Millipore). The percentage of fluorescent siRNA (Cy5) was measured in the ultrafiltrate using a fluorescence-based microplate reader at a wavelength of excitation 650 nm and emission 670 nm. A standard curve was used to determinate the amount of siRNA from the fluorescence intensity.

Evaluation of the Retardation of miRNA by CLCNs:

9 μl of CLCNs complexed with siRNA Cy5 was mixed with 1 μl of loading buffer (6×DNA Loading Thermo Scientific). The samples were loaded into a 1% (w/v) agarose gel containing 0.5 μg/ml ethidium bromide per well. Electrophoresis was carried out at 100 V for 20 minutes in Tris-acetate-EDTA (TAE) running buffer. After electrophoresis, the gel was analyzed with use of a gel imaging system. The relative density of the bands was calculated using ImageJ software.

Nanoparticle Tracking Analysis (NTA):

Nanoparticle size and concentration were measured at the same time by using a NanoSight NS300 Instrument (Malvern Instruments). A fluorescence mode provides differentiation of labeled or naturally fluorescing nanoparticles. The instrument uses a particle-by-particle system to produce high-resolution results for particle size, distribution, and concentration. The standard nanoparticle concentration in a diluted sample volume of ˜1 ml was estimated to be about 10⁶-10⁹ particles/ml.

Cellular Uptake and Processing of CLCNs in H1299.

In vitro cellular uptake and processing of CLCNs were evaluated in tumor cell line H1299 by using quantitative and imaging methodologies. H1299 cells were seeded into a 6-well plate at 2×10⁵ cells/well and cultured overnight. D275 fluorescent CLCNs (CLCNs D275 wavelength 460/580 nm) and CLCNs conjugated with Cy5 siRNA (CLCNs-siRNA Cy5 wavelength 650/670 nm) at a concentration of 100 nM were incubated with H1299 cells for 24 hours, and uptake was evaluated by fluorescence microscopy (with an Olympus IX81 microscope) or flow cytometry (Gallios Flow Cytometer). In fluorescence and confocal microscopy experiments, H1299 cells were seeded into a 4-chamber slide (Nunc™ LabTek™ II Chamber Slide™ System, ThermoFisher Scientific) at 2×10³. After 24 hours, cells were treated with CLCNs D275 or CLCNs-siRNA Cy5 for 2, 4, 6, 8, and 24 hours. For fluorescence microscopy images the nuclei were stained with DAPI (ThermoFisher Scientific) and the ER was stained with ER-Tracker™ (fluorescence wavelength of 587/615) (ThermoFisher Scientific). For confocal microscope analyses, H1299 cells were fixed with PFA 4% and the nuclei were stained with DAPI. Confocal images were taken using a FV 1000 Olympus Laser Confocal.

Transmission Electron Microscope:

Transmission electron microscope images (JEM-1010 Transmission Electron Microscope) were acquired to evaluate the morphology and the structure of CLCNs and the internalization of the CLCNs in the tumor cells at various time points (2, 4, 6, 8, 12, and 24 hours). A small drop of the CLCN formulations was deposited on the carbon coated grid, allowed to settle, blotted dry and then covered with a small drop of the negative stain. For the in vitro experiments H1299 cells were seeded in 35-mm culture dishes, treated after 24 hours with CLCN formulations, incubated with a fixative solution (2.5% glutaraldehyde in 0.1 M sodium cacodylate), and stored at 4° C. A small drop of the cell suspensions was deposited on the carbon coated grid and covered with a small drop of the negative stain. The images were acquired with use of a JEOL JEM-1010 transmission electron microscope equipped with digital cameras.

GFP Silencing Assay:

H1299 cells were seeded 2×10⁵ in 6-well tissue culture plates in triplicate and grown overnight at 37° C. with 5% CO₂. When 80% confluent, cells were transfected with 2.5 μg of GFP plasmid (pMAX-GFP) using Lipofectamine transfection reagent (Invitrogen). In a first experiment cells where cotransfected with GPF Plasmid-Lipofectamine and CLCNs-anti-GFP Positive Control siRNA (siGFP) or CLCNs-negative control siRNA (Ambion® Silencer GFP) at a concentration of 100 nM. After 24 hours, cells were analyzed by fluorescence microscopy (Olympus IX81) and flow cytometry (Gallios Flow Cytometer). To this purpose, cells were washed with PBS, trypsinized, washed once with flow cytometry washing solution (PBS 3% FBS), and analyzed by flow cytometry. In another experiment anti-GFP Positive Control siRNA (siGFP) or negative control siRNA (Ambion® Silencer GFP) at a concentration of 100 nM were delivered by either CLCNs or DharmaFECT transfection reagents (Dharmacon) to compare silencing efficiency. After 24 hours, cells were analyzed by fluorescence microscopy (Olympus IX81) and the fluorescence intensity was calculated using, ImageJ software. GFP silencing was calculated as the percentage of GFP fluorescence intensity in samples treated with CLCNs-siRNA anti-GFP compared with control samples.

Gene Expression In Vitro:

Cells were seeded in 6-well plates at an initial density of 2×10⁵ cells/well. After 24 hours, the cells were treated with CLCNs conjugated with miR30b (Ambion) or DharmaFECT transfection reagents (Dharmacon) mixed with miR30b as well. Various concentrations of miR30b (25, 50, and 100 nM) were used and the same concentration were used for the NSC-siRNA (negative control) conjugated to CLCNs or to DharmaFect. After 24 hours total RNA was extracted from the samples by using TRIzol® RNA Isolation Reagents (Thermo Fisher Scientific). Reverse transcription was performed by using a High-Capacity cDNA Reverse Transcription Kit (Applied Biosystems) and miR30b RT primers. Quantitative real-time PCR (qPCR) was performed by using TaqMan® MicroRNA Assays (Life Technologies) on a CFX384 Real-Time System (Bio-Rad) and miR30b TM primers.

In Vitro Cytotoxicity Assay:

Cells were seeded in 96-well plates at an initial density of 3×10³ cells/well. After 24 hours, cells were treated with various concentrations of CLCNs and CLCNs/siRNA Cy5, for 24, 48, and 72 hours at 37° C. in a humidified, 5% CO₂ atmosphere. The cytotoxicity at each time point was evaluated by using a standard 2,3-bis (2-methoxy-4-nitro-5-sulfophenyl)-5-[(phenylamino) carbonyl]-2H-tetrazolium hydroxide (XTT) II assay (Sigma-Aldrich). Absorbance was determined on a plate reader at 492 nm. The percentage of cell viability was calculated according to the following equation:

$$cell viability ( \% ) = \frac{ABS T}{ABS C} \times 100$$

Where ABS T is the absorbance of treated cells and ABS C is the absorbance of control (no treated) cells.

In Vivo Studies:

Animal studies were approved by the Institutional Animal Care and Use Committee of The University of Texas M. D. Anderson Cancer Center and performed according to NIH guidelines. Female nude mice (nu/nu), aged 4-6 weeks, were purchased from the Charles River Company. Before any experiment were started, the mice were acclimatized for 5 days in the animal core facility.

In Vivo Fluorescent CLCNs D275 Biodistribution:

H1299 cells were injected into nu/nu female mice aged 4-6 weeks at 1×10⁶ cells/mouse via subcutaneous injection on the right flank. After about 3 weeks, the tumor size was approximately 1 cm. Mice were randomized and divided in 3 different groups: no treatment, CLCN1 D275, and CLCN2 D275. Green fluorescence CLCNs were administered intravenously at 10 mg/kg via tail vein injection. After 24 hours, the mice were euthanized, and tumor, and major organs (liver, spleen, brain, lung, and kidney) were collected. The fluorescent signal of CLCNs in organs and tumor was detected by fluorescence microscopy and flow cytometry analysis. Briefly, for fluorescence microscopy studies, the whole tissue was embedded in OCT medium and frozen in dry ice; 5- to 15-μm-thick sections were cut at −20° C. and transferred to a microscope slide at room temperature. The slides were imaged with use of a fluorescence microscope (LEICA DM5500 B) equipped with a FITC filter to visualize the green fluorescence of CLCNs. For flow cytometry analysis, organs and tissues were mechanically disaggregated by using 70-1 μm and 35-μm cell strainers to generate a single-cell suspension in PBS and analyzed by flow cytometry as described above.

In Vivo CLCNs-miR30b Biodistribution:

The 4- to 6-week-old nu/nu female mice bearing H1299 subcutaneous tumors were treated with CLCNs-miR30b and CLCNs-negative siRNA control at 1.5 mg/kg via tail vein injection. After 24 hours, the mice were euthanized, and tumors and major organs (liver, spleen, brain, lung, and kidney) were collected and stored in RNAlater solutions for RNA stabilization at −80° C. Total RNA was extracted from tissue samples by using TRIZOL® RNA Isolation Reagents (Thermo Fisher Scientific). Reverse transcription was performed by using a High-Capacity cDNA Reverse Transcription Kit (Applied Biosystems) and miR30b RT primers. Quantitative real-time PCR (qPCR) was performed by using TaqMan MicroRNA Assays (Life Technologies) on a CFX384 Real-Time System (Bio-Rad) and miR30b TM primers.

Chemical and Histological Analysis of Blood and Major Organs:

Nu/nu female mice bearing subcutaneous tumors on the right flank, were randomized and divided into 3 different groups: no treatment, CLCN1 D275, and CLCN2 D275. Green fluorescence CLCNs were administered intravenously at 10 mg/kg via tail vein injection. After 24 hours, the mice were euthanized, and blood, tumor, and major organs (liver, spleen, brain, lung, and kidney) were collected. Blood was tested for liver or kidney function alteration by routine chemical analysis. The whole tissue was embedded in OCT medium and frozen in dry ice; 5- to 15-μm-thick sections were cut at −20° C. and transferred to a microscope slide at room temperature. Sections were stained for hematoxylin and eosin (H & E). The tissue sections were evaluated by a pathologist (AP) without knowledge of the treatment groups.

In Vivo CLCN2-miR50 Inhibitor Systemic Administration:

H1299 cells were injected into the flank of 6-8-week old nu/nu mice. After 2 weeks all mice developed a subcutaneous tumor and were randomized and divided in 2 groups (n=5 mice each group). One group was treated for one week with 3 intravenous injections by tail vein of CLCN2-miR150 inhibitor at 1.5 mg/kg and one group was used as a control group. Tumors were measured 3 times a week for 3 weeks, and the growth rate was compared among the two treatment groups using generalized linear regression models to account for inter-mouse variability and the longitudinal nature of the data.

Statistical Analysis:

All the numeric data are the result of a minimum of three independent experiments. Statistical computation was performed with Prism GraphPad software. The statistical significance was calculated with use of a two-tailed unpaired Student t test. SAS version 9.4 and S-Plus version 8.04 are used to carry out the computations for in vivo data analyses.

### Example 3—Immune Cell Transfection with CLCNs

T-Cells Uptake and Internalization of CLCN-RNAi In Vitro:

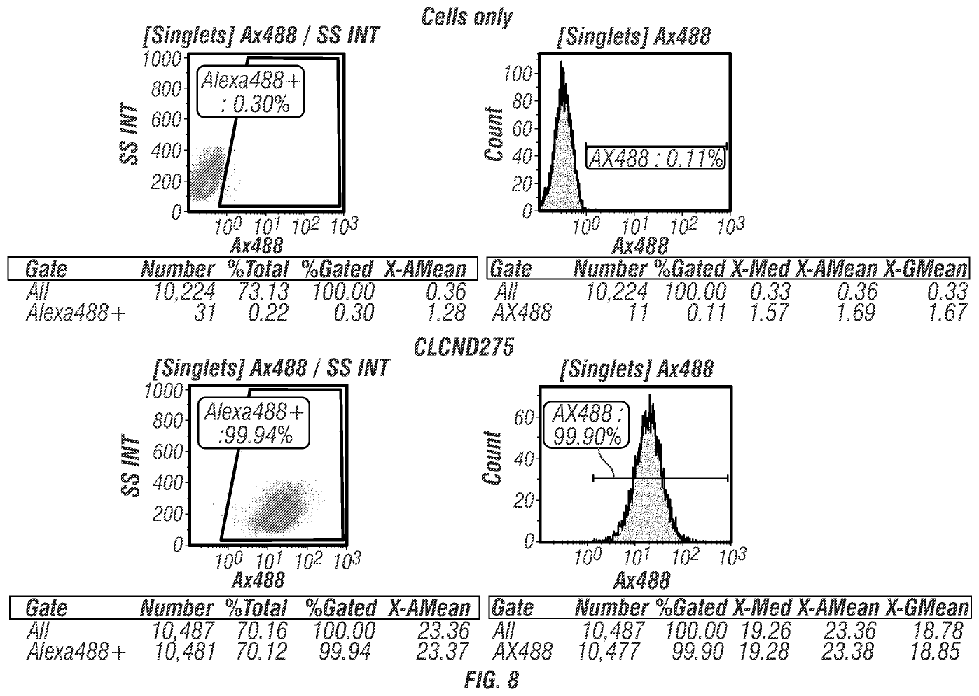

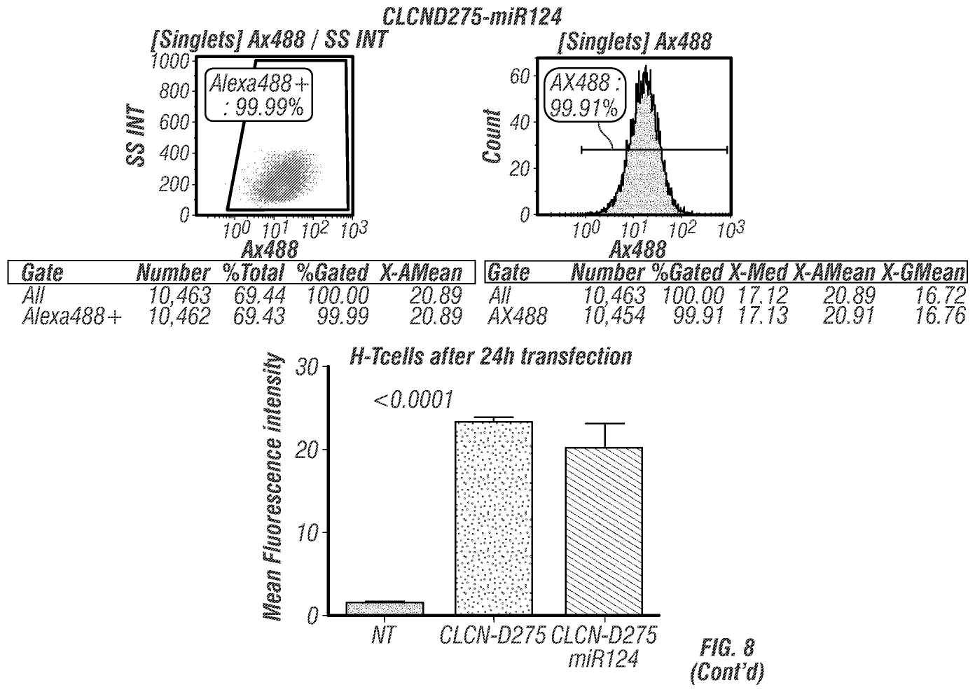

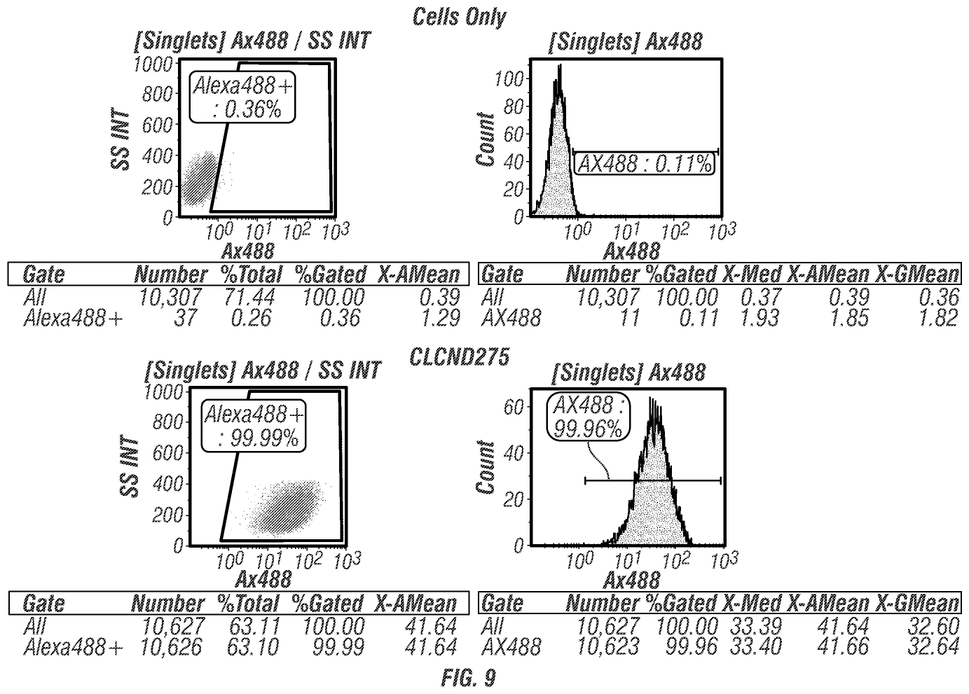

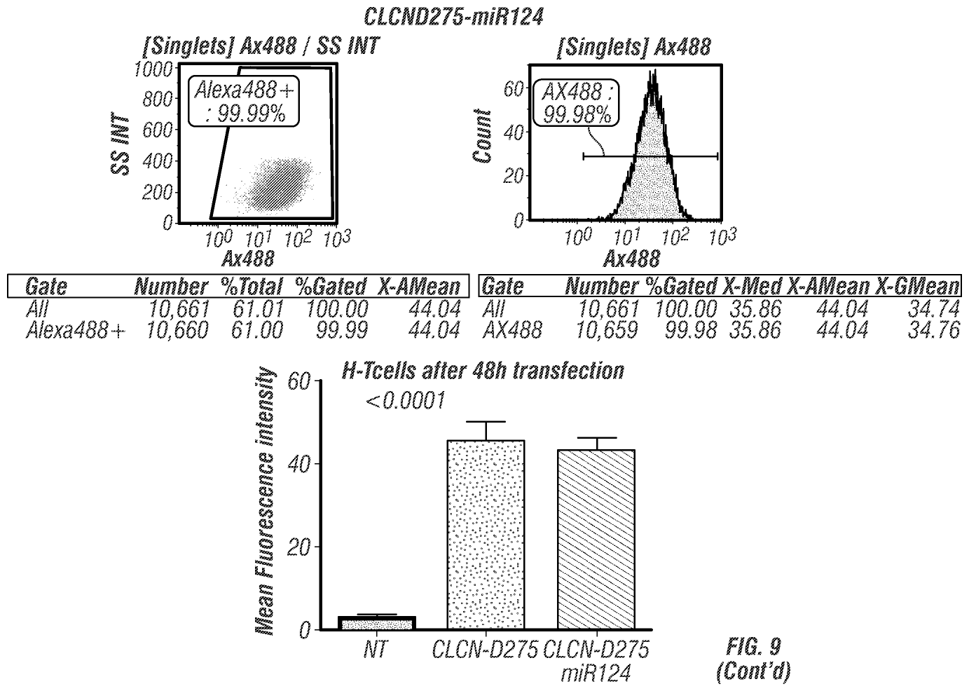

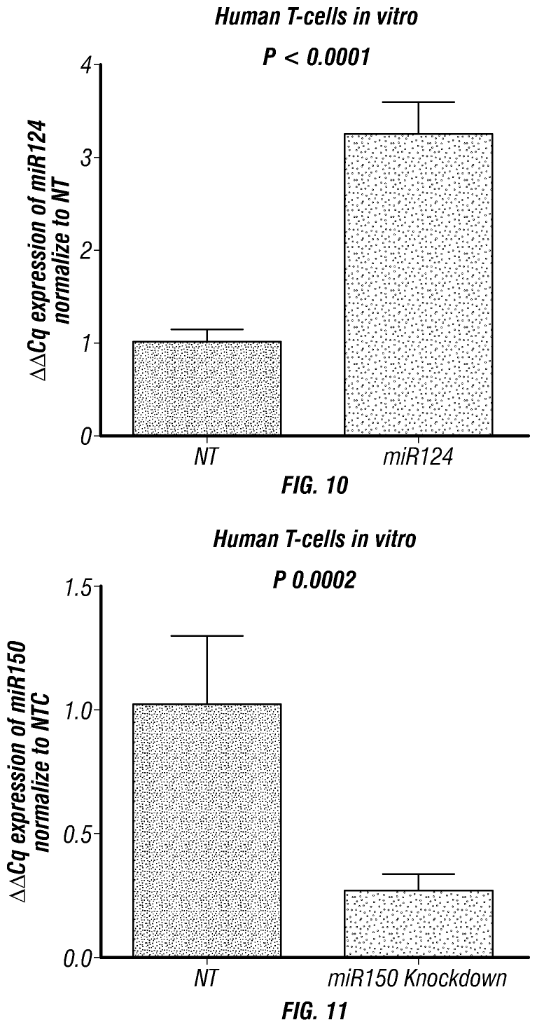

Cellular uptake and internalization of CLCN-RNAi nanoparticles were evaluated in Human T-cells by Flow Cytometry (FIG. 8). Human T-cells were treated for 24 and 48 hours with Green fluorescent labeled CLCNs (CLCN D275) (FIGS. 9 and 10). At the end of each time point the cells were collected and the fluorescent intensity was evaluated by Flow cytometry at the wavelength of ex 484 and em 501.

In Vitro Transfection Efficiency Evaluation:

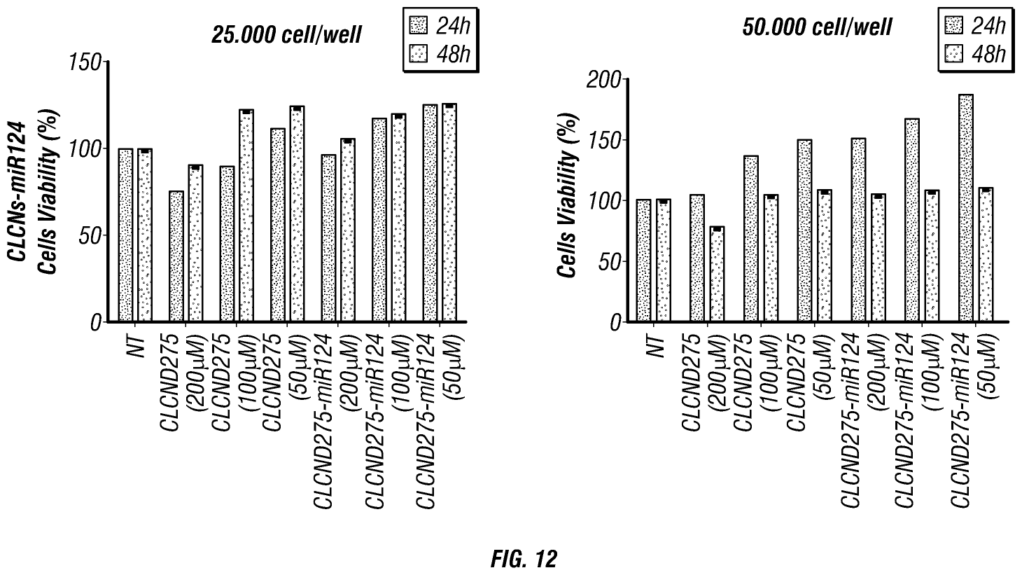

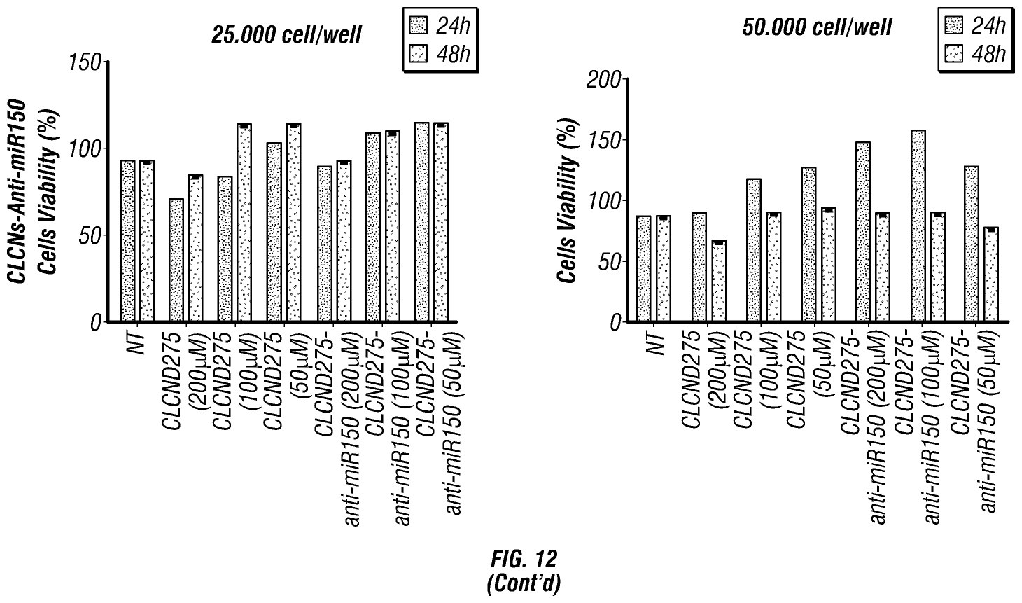

Human T-cells were treated for 24 hours with CLCNs conjugated with miR124 (FIG. 10) and CLCNs-150 inhibitor (FIG. 11). The CLCNs transfection efficiency was evaluated in Human T-cells using a TAQMAN® MicroRNA Assays in a Quantitative real-time PCR (qPCR). Human T-cells were transfected with CLCNs-miR124 at the concentration of 100 nM and after 24 hours the relative gene expression of miR124 was calculated (FIG. 10). U6 was used as reference gene. In another experiment Human T-cells were treated with CLCNs conjugated with a miR150 inhibitor at the concentration of 25 nM and after 24 hours the knockdown of miR150 was evaluated (FIG. 12). GAPDH was used as reference gene. The relative expression of miR124 or miR150 was normalized to the no treated group.

Evaluation of Cytotoxicity of CLCNs on Human T-Cells:

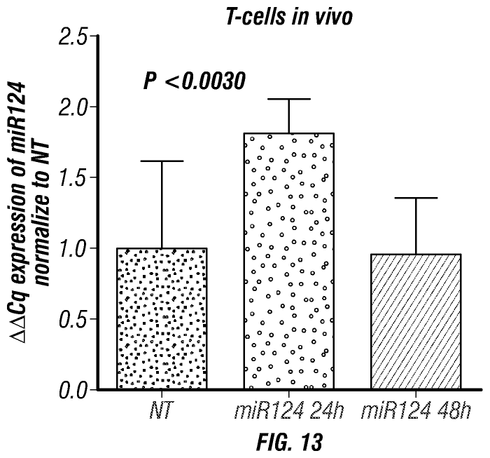

Cytotoxicity of CLCNs alone or complexed with synthetic RNAi molecules was assessed in Human T-cells by XTT proliferation assay (FIG. 13). Human T-cells were seeded in 96 well plates at different cell density, 25.000 cells/well and 50.000 cells/well. Different concentrations of CLCNs alone and conjugated with miR124 or miR150 inhibitor, were used (200, 100 and 50 μM). The XXT assay was performed 24 and 48 hours after the treatment.

Evaluation of CLCN-miR124 Delivery to T-Cells after Systemic Injection:

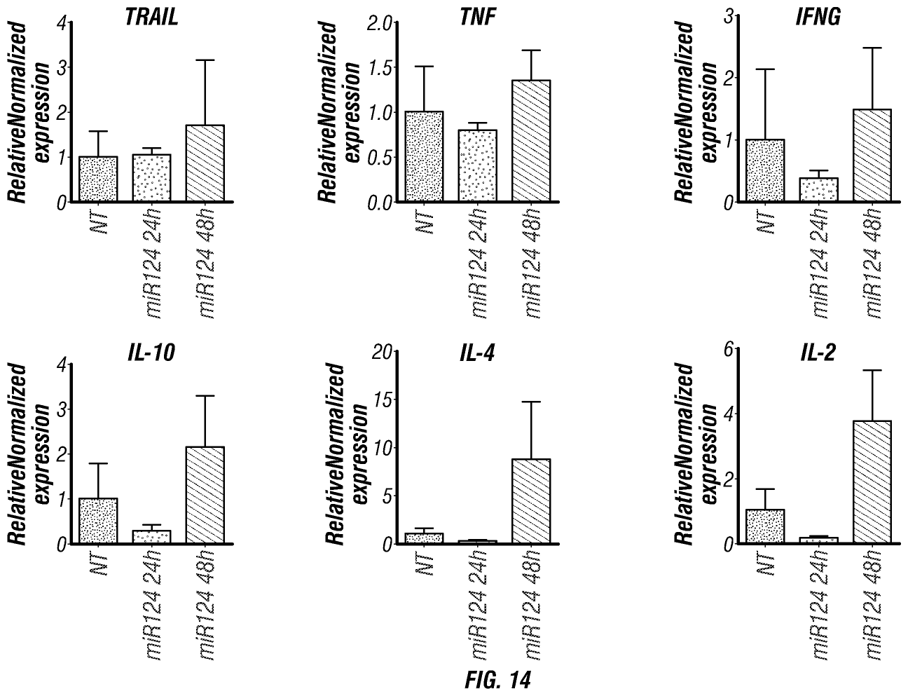

In vivo experiments were performed to evaluate the ability of CLCNs to deliver miR124 to T-cells after systemic injection. C57BL/6J mice were divided in 3 different groups (5 mice each group) and systemically injected by tail vein with CLCN-miR124 at a dose of 1.2 mg/Kg. After 24 hours and 48 hours the spleen was collected and a single-cell suspensions was made. The single cell suspension was stained with CD3 antibodies and the T-cells were sorted. The relative expression of miR124 was measured by RNA extraction and a TAQMAN® MicroRNA Assays in a Quantitative real-time PCR (qPCR). U6 was used as reference gene (FIG. 14).

Cytokines Expression in T-Cells after Treatment with CLCNs-miR/24 In Vivo for 24 and 48 Hours:

The relative expression of some of the important cytokines such as IL10, IL2 and IL4 and T-cells co-stimulatory factors such as TRAIL. TNF. IFN GAMMA, was quantified with RT-qPCR (FIG. 15). Briefly, C57BL/6J mice were systemically injected by tail vein with CLCNs-miR124 at the concentration of 1.5 mg/kg. The spleens were collected 24 and 48 hours after treatment. A single-cell suspension was made from spleen and T-cells were sorted. The total RNA was extracted from the sorted T-cells and the expression of the selected cytokines and costimulatory factors was evaluated by RT-qPCR.

All of the compositions and methods disclosed and claimed herein can be made and executed without undue experimentation in light of the present disclosure. While the compositions and methods of the present disclosure have been described in terms of preferred embodiments, it will be apparent to those of skill in the art that variations may be applied to the methods and in the steps or in the sequence of steps of the method described herein without departing from the concept, spirit and scope of the present disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the present disclosure as defined by the appended claims.

### REFERENCES

The following references, to the extent that they provide exemplary procedural or other details supplementary to those set forth herein, are specifically incorporated herein by reference.
Filipe V et al., Pharmaceutical Research, 27: 796-810, 2010.
International Patent Publication No. WO1995001994
International Patent Publication No. WO1998042752
International Patent Publication No. WO2000037504
International Patent Publication No. WO2001014424
International Patent Publication No. WO2009/101611
International Patent Publication No. WO2009/114335
International Patent Publication No. WO2010/027827
International Patent Publication No. WO2011/066342
International Patent Publication No. WO2015016718
Lv H et al., Journal of Controlled Release, 114: 100-9, 2006.
Remington's Pharmaceutical Sciences, 18th Ed. Mack Printing Company, 1990.
U.S. Pat. No. 4,870,287
U.S. Pat. No. 5,760,395
U.S. Pat. No. 5,844,905
U.S. Pat. No. 5,885,796
U.S. Pat. No. 6,207,156
U.S. Pat. No. 6,673,611
U.S. Pat. No. 8,008,449
U.S. Pat. No. 8,017,114
U.S. Pat. No. 8,119,129
U.S. Pat. No. 8,329,867
U.S. Pat. No. 8,354,509
U.S. Pat. No. 8,735,553
U.S. Patent Publication No. 20040019001
U.S. Patent Publication No. 20110008369
U.S. Patent Publication No. 20140294898

What is claimed is:

1. A cationic liquid crystalline nanoparticle (CLCN) comprising glycerol monooleate (GMO), a cationic phospholipid, and a nonionic surfactant present in a concentration of up to 5% by weight, wherein the nanoparticle is positively charged.

2. The nanoparticle of claim 1, wherein the nanoparticle comprises a lipid bilayer enclosing an aqueous core, wherein the bilayer is surrounded by a hydrophobic shell.

3. The nanoparticle of claim 1, wherein the nonionic surfactant is present at a concentration of 0.1 to 1% by weight.

4. The nanoparticle of claim 1, wherein the nonionic surfactant is present at a concentration of 0.5% by weight.

5. The nanoparticle of claim 1, wherein the nonionic surfactant is a nonionic polyol.

6. The nanoparticle of claim 5, wherein the nonionic polyol is tri-block polyethylene glycol-polypropylene-polyethylene glycol.

7. The nanoparticle of claim 5, wherein the nonionic polyol is PLURONIC® F-127.

8. The nanoparticle of claim 1, wherein the nanoparticle has a zeta potential greater than +30 mV.

9. The nanoparticle of claim 1, wherein the nanoparticle has a zeta potential of +25 to +35 mV.

10. The nanoparticle of claim 1, wherein the nanoparticle has a diameter of 60 to 100 nm.

11. The nanoparticle of claim 1, wherein the cationic phospholipid is selected from the group consisting of 2-dioleoyl-3-trimethylammonium-propane chloride salt (DOTAP), Dimethyldioctadecylammonium (DDAB), 1,2-di-O-octadecenyl-3-trimethylammonium propane (DOTMA), 3ß-[N—(N′,N′-dimethylaminoethane)-carbamoyl]cholesterol hydrochloride (DC-CHOL), 1,2-dioleoyl-3-dimethylammonium-propane (DODAP), cetyl trimethyl ammonium bromide (CTAB), 1,3-dioleoyloxy-2-(6-carboxy-spermyl)-propyl-amide (DOSPER), and 1,2-dioleoyl-sn-glycero-3-[(N-(5-amino-1-carboxypentyl)iminodiacetic acid)succinyl](DOGS).

12. The nanoparticle of claim 1, wherein the cationic phospholipid is DOTAP.

13. The nanoparticle of claim 1 or claim 12, wherein the cationic phospholipid is present at a concentration of less than 30% by weight.

14. The nanoparticle of claim 1 or claim 12, wherein the cationic phospholipid is present at a concentration of less than 20% by weight.

15. The nanoparticle of claim 1 or claim 12, wherein the cationic phospholipid is present at a concentration of less than 15% by weight.

16. The nanoparticle of claim 1 or claim 12, wherein the cationic phospholipid is present at a concentration of 5-10% by weight.

17. The nanoparticle of claim, wherein the glycerol monooleate is 1-(cis-9-octadecenoyl)-rac-glycerol.

18. The nanoparticle of claim 1, wherein the nanoparticle has a polydispersity index (PDI) of 0.10 to 0.20.

19. The nanoparticle of claim 1, wherein the nanoparticle is loaded with RNA.

20. The nanoparticle of claim 19, wherein the RNA is siRNA or miRNA.

21. The nanoparticle of claim 19, wherein the RNA has a length of 15 to 50 nucleotides.

22. The nanoparticle of claim 19, wherein the RNA is loaded in the nanoparticle at a 1:1 volume ratio.

23. The nanoparticle of claim 19, wherein the RNA is encapsulated within the nanoparticle.

24. The nanoparticle of claim 19, wherein the zeta potential of the RNA-loaded nanoparticle is +30 to +45 mV.

25. A composition comprising a plurality of nanoparticles of any one of claims 1-24.

26. The composition of claim 25, wherein the nanoparticles have a MMAD of 60-100 nm.

27. A pharmaceutical composition comprising a plurality of nanoparticles of any one of claims 1-24 in combination with a pharmaceutically acceptable carrier.

28. A method of producing cationic liquid crystalline nanoparticles (CLCNs) comprising:
(a) solubilizing comprising a cationic phospholipid and glycerol monooleate in ethanol to obtain a lipophilic stage;
(b) solubilizing a nonionic surfactant in water to obtain a hydrophilic phase;
(c) emulsifying the lipophilic phase and the hydrophilic phase using high-speed homogenization to obtain a nanoparticle solution; and
(d) evaporating the ethanol from the nanoparticle solution, thereby obtaining CLCNs.

29. The method of claim 28, wherein the CLCNs are the CLCNs of any one of claims 1-24.

30. The method of claim 28, wherein the nonionic surfactant is Pluronic F-127.

31. The method of claim 28, wherein step (c) is further defined as dropwise addition of the hydrophilic phase to the lipophilic phase, wherein the lipophilic phase is under high-speed homogenization.

32. The method of claim 27, wherein high-speed homogenization is at a speed of 7,000-10,000 rpm.

33. The method of claim 28, further comprising applying the CLCN solution of step (c) to one or more rounds of high-speed homogenization.

34. The method of claim 33, wherein the high-speed homogenization is at a speed of 10,000 to 20,000 rpm.

35. The method of claim 28, wherein evaporating ethanol comprises subjecting the CLCN solution to magnetic stirring for at least 15 hours.

36. The method of claim 28, further comprising encapsulating RNA into the CLCNs.

37. The method of claim 24, wherein encapsulating comprises adding an RNA solution to the CLCNs and vortexing to obtain RNA-loaded CLCNs.

38. The method of claim 28, wherein the RNA is siRNA or miRNA.

39. The method of claim 37, wherein the RNA is added at a 1:1 volume ratio of CLCNs:RNA.

40. The method of claim 37, wherein at least 75% of the RNA is encapsulated into the CLCNs.

41. The method of claim 28, wherein the method does not comprise chloroform, the formation of lipophilic film, or sonication.

42. Cationic liquid crystalline nanoparticles (CLCNs) produced by a method in accordance with any one of claims 28-41.

43. A method of delivering an RNA into a cell comprising administering an effective amount of RNA-loaded CLCNs of any one of claims 19-24 to the cell.

44. The method of claim 43, wherein the RNA-loaded CLCNs are produced according any one of claims 28-41.

45. The method of claim 43, wherein the cell is a human cell.

46. The method of claim 45, wherein the cell is a cancer cell or a T cell.

47. A method of treating a disease or disorder in subject in need thereof comprising administering an effective amount of CLCNs of any one of claim 1-24 to the subject.

48. The method of claim 47, wherein the CLCNs are loaded with siRNA or miRNA.

49. The method of claim 47, wherein the disease or disorder is cancer, an inflammatory disorder, or an immune-associated disorder.

50. The method of claim 49, wherein the cancer is lung cancer.

51. The method of claim 50, wherein the CLCNs are loaded with miR150 inhibitor.

52. The method of claim 47, wherein the subject is a human.

53. The method of claim 49, wherein the CLCNs are administered orally, topically, intravenously, intraperitoneally, intramuscularly, endoscopically, percutaneously, subcutaneously, regionally, or by direct injection.

54. The method of claim 49, wherein the CLCNs are administered intravenously.

55. The method of claim 49, further comprising administering at least a second therapeutic agent.

56. The method of claim 55, wherein the at least a second therapeutic agent is an anti-cancer agent.

57. A method of immunostimulating a subject comprising administering an effective amount of CLCNs of any one of claim 1-24 to the subject, wherein the CLCNs are loaded with immune-modulatory RNA.

58. The method of claim 57, wherein the CLCNs are delivered to T cells.

59. The method of claim 58, wherein the CLCNs result in an altered cytokine profile.
